(19) United States
(12) Patent Application Publication
(10) Pub. No.: US 2023/0329912 A1
(43) Pub. Date: Oct. 19, 2023

(54) NEW TINNITUS MANAGEMENT TECHNIQUES

(71) Applicant: COCHLEAR LIMITED, Macquarie University, NSW (AU)

(72) Inventors: Alexander VON BRASCH, Macquarie University (AU); Stephen FUNG, Macquarie University (AU)

(21) Appl. No.: 18/025,523

(22) PCT Filed: Sep. 9, 2021
(86) PCT No.: PCT/IB2021/058210

§ 371 Date: Mar. 9, 2023

Related U.S. Application Data
(60) Provisional application No. 63/076,078, filed on Sep. 9, 2020.

(51) Int. Cl. A61F 11/00; A61B 5/12; G16H 40/63; G16H 50/20; G16H 50/70; G16H 20/30

(52) U.S. Cl. CPC A61F 11/00; A61B 5/128; G16H 40/63; G16H 50/20; G16H 50/70; G16H 20/30

(57) ABSTRACT

A method, including automatically obtaining data indicative of at least one of physiological features past and/or present of a person who experiences recurring tinnitus or ambient environmental conditions past and/or present of the person, analyzing the obtained data to determine at least one of that a tinnitus event is occurring or that a tinnitus event has a statistical likelihood of occurring in the near term and initiating a tinnitus mitigation method based on the action of analyzing.

### CROSS-REFERENCE TO RELATED APPLICATIONS

This application claims priority to U.S. Provisional Application No. 63/076,078, entitled NEW TINNITUS MANAGEMENT TECHNIQUES, filed on Sep. 9, 2020, naming Alexander VON BRASCH of Macquarie University, Australia as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have performed lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a method, comprising automatically obtaining data indicative of at least one of physiological features past and/or present of a person who experiences recurring tinnitus or ambient environmental conditions past and/or present of the person, analyzing the obtained data to determine at least one of that a tinnitus event is occurring or that a tinnitus event has a statistical likelihood of occurring in the near term, and initiating a tinnitus mitigation method based on the action of analyzing.

In an exemplary embodiment, there is an apparatus, comprising a body carried portable device including an input subsystem and an output subsystem, wherein the device includes a product of and/or resulting from machine learning that is used by the device to determine when and/or if to initiate a tinnitus management action.

In an exemplary embodiment, there is a method, comprising logging first data corresponding to least one of physiological features past and/or present of a person who experiences recurring tinnitus or ambient environmental conditions past and/or present of the person, logging second data corresponding to tinnitus related events and/or non-events, correlating the logged first data with the logged second data utilizing a machine learning system and developing, with the machine learning system, a tinnitus management regime.

In an exemplary embodiment, there is a system, comprising a sound capture apparatus configured to capture ambient sound and an electronics package configured to receive data based on at least an outputted signal from the sound capture apparatus and analyze the data to determine based thereon that there exists a statistical likelihood of a future tinnitus event in the near term of a person using the system, wherein the system is configured to automatically initiate an output that preemptively reduces the likelihood of the future tinnitus event upon the determination. In an exemplary embodiment, there is a system, comprising a tinnitus onset predictive subsystem and a tinnitus management output subsystem.

### BRIEF DESCRIPTION OF THE DRAWINGS

Embodiments are described below with reference to the attached drawings, in which:

FIG. 1 is a perspective view of an exemplary hearing prosthesis in which at least some of the teachings detailed herein are applicable;

FIGS. 1A-1C are quasi functional diagrams of an exemplary device to which some embodiments may be applicable;

FIGS. 1D-2C present exemplary devices and/or systems that can be used to execute at least some of the teachings herein;

FIGS. 3-5, 7B and 7C present exemplary flowcharts for some exemplary methods; and

FIGS. 6, 7, 7A, 8, 9 and 10 present functional diagrams for some exemplary embodiments.

### DETAILED DESCRIPTION

Merely for ease of description, the techniques presented herein are primarily described herein with reference to an illustrative medical device, namely a hearing prosthesis. First introduced is a bimodal hearing prosthesis that includes a cochlear implant and an acoustic hearing aid (a multimode hearing prosthesis). The techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant and/or an acoustic hearing aid, corresponds to a disclosure of another embodiment of using such teaching with another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, electroporation, etc., and thus any disclosure herein is a disclosure of utilizing such devices with the teachings herein, providing that the art enables such. The teachings herein can also be used with conventional hearing devices, such as telephones and ear bud devices connected MP3 players or smart phones or other types of devices that can provide audio signal output. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

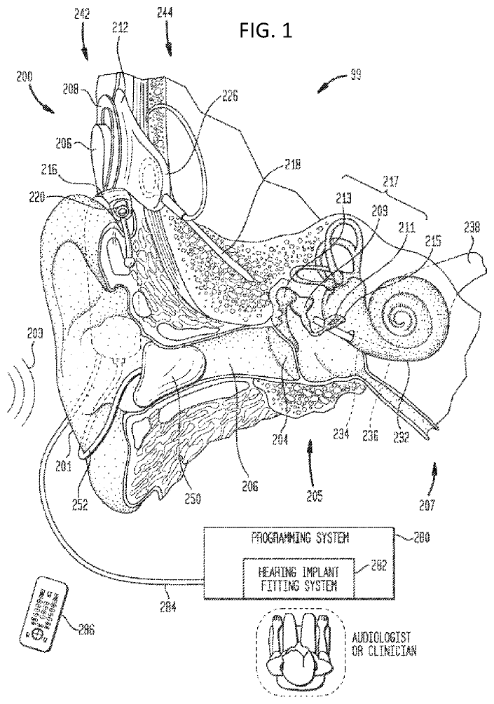

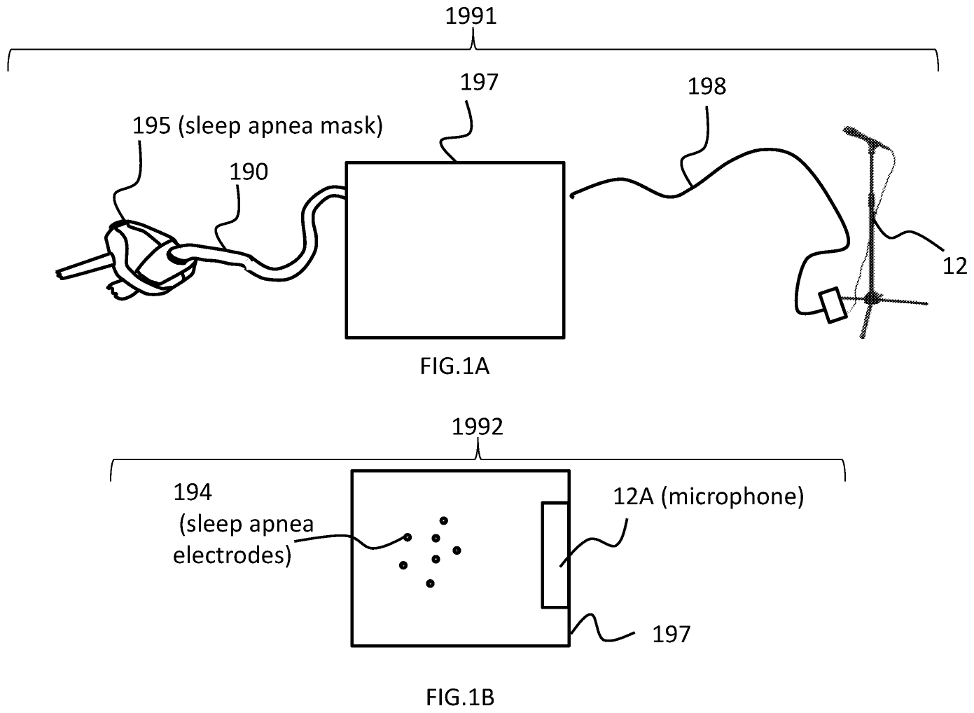

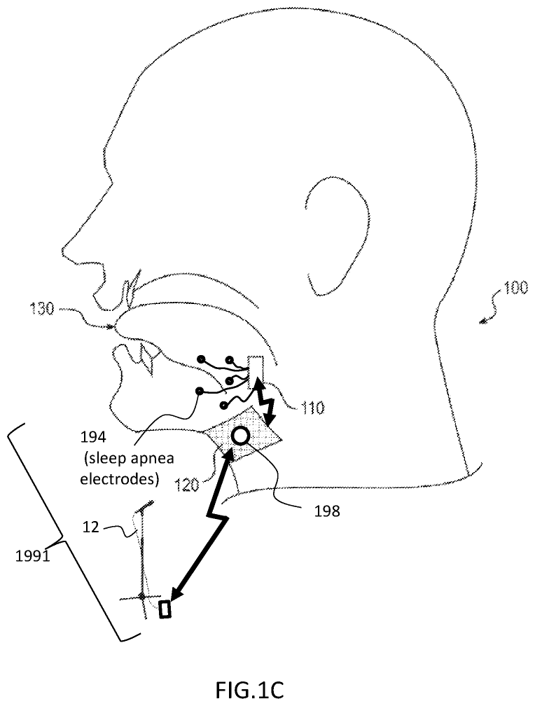

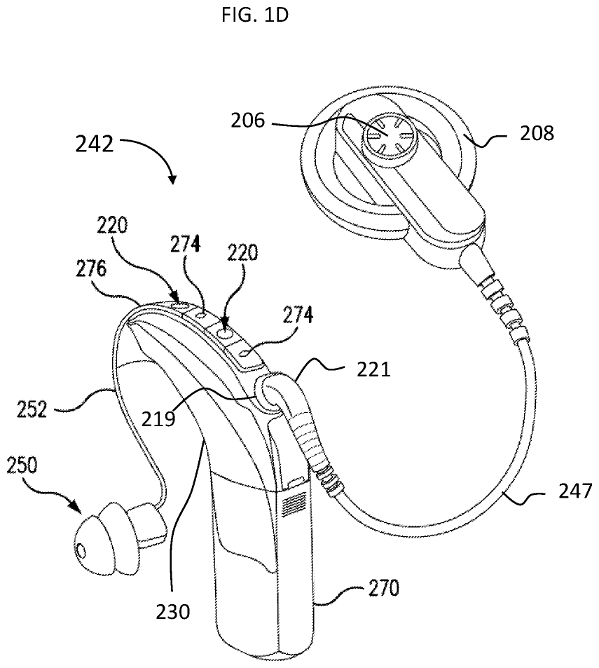

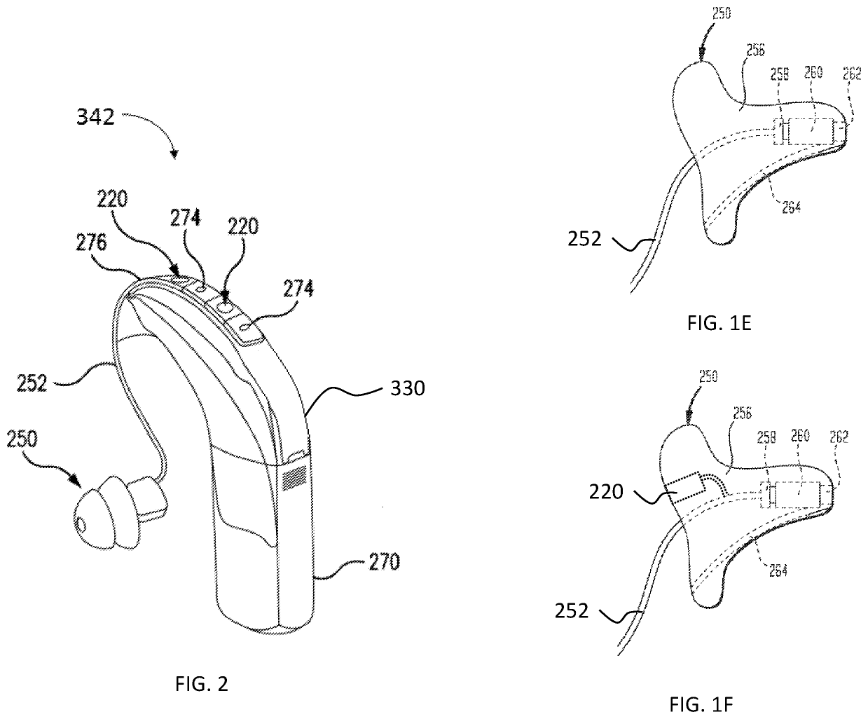

FIG. 1 is a perspective view of an exemplary multimodal prosthesis. The ear includes outer ear 201, middle ear 205, and inner ear 207, and are described next below, followed by a description of an implanted multimodal system 200. Multimodal system 200 provides multiple types of stimulation, i.e., acoustic, electrical, and/or mechanical. These different stimulation modes may be applied ipsilaterally or contralaterally. In the arrangement shown in FIG. 1, multimodal implant 200 provides acoustic and electrical stimulation, although other combinations of modes can be implemented in some embodiments. By way of example and not by way of limitation, a middle-ear implant can be utilized in combination with the cochlear implant, a bone conduction device can be utilized in combination with the cochlear implant, etc.

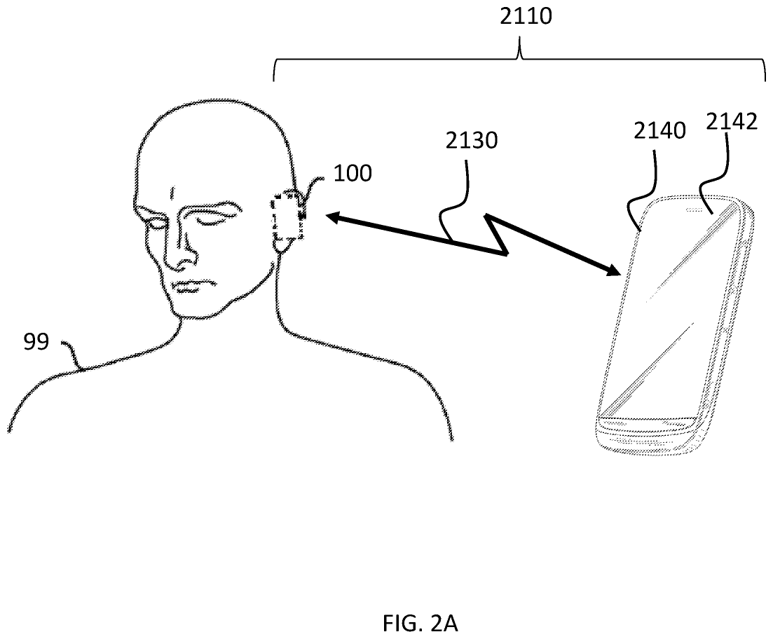

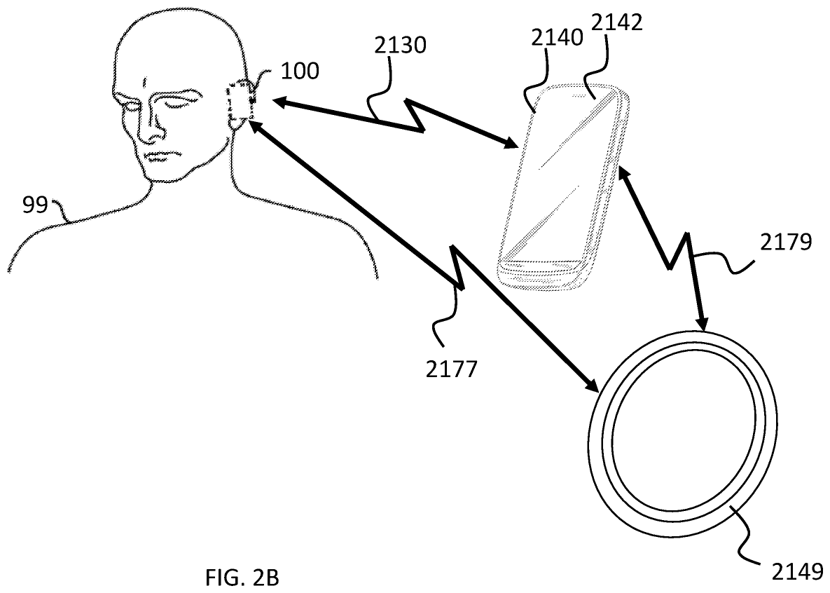

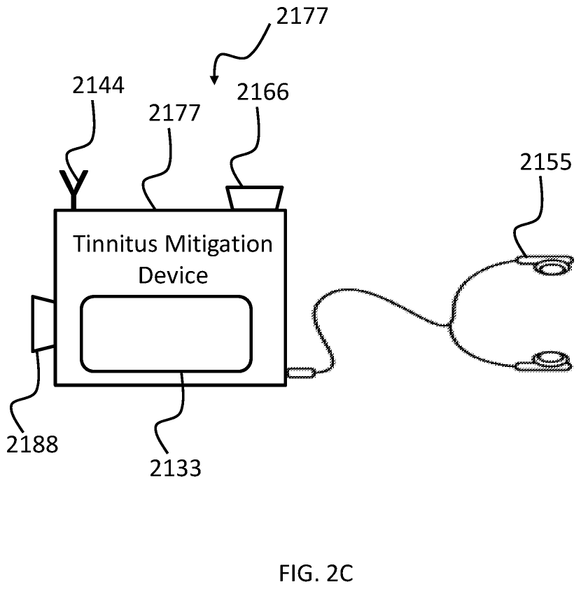

It is also noted that embodiments are directed to a purely acoustic hearing aid, as detailed below in FIG. 2. That said, embodiments are directed to non-hearing aid per se devices, but instead tinnitus masking devices that utilize some aspects of hearing aids, and in other embodiments, do not use such aspects. Indeed, some embodiments are directed to pure tinnitus maskers. Some embodiments can be implemented in conventional earphones/ear buds, telephones, etc. Thus any teaching herein corresponds to an embodiment where one or more or all of the teachings herein are utilized in such devices.

In a person with normal hearing or a recipient with residual hearing, an acoustic pressure or sound wave 203 is collected by outer ear 201 (that is, the auricle) and channeled into and through ear canal 206. Disposed across the distal end of ear canal 206 is a tympanic membrane 204 which vibrates in response to acoustic wave 203. This vibration is coupled to oval window, fenestra ovalis 215, through three bones of middle ear 205, collectively referred to as the ossicles 217 and comprising the malleus 213, the incus 209, and the stapes 211. Bones 213, 209, and 211 of middle ear 205 serve to filter and transfer acoustic wave 203, causing oval window 215 to articulate, or vibrate. Such vibration sets up waves of fluid motion within cochlea 232. Such fluid motion, in turn, activates tiny hair cells (not shown) that line the inside of cochlea 232. Activation of the hair cells causes appropriate nerve impulses to be transferred through the spiral ganglion cells (not shown) and auditory nerve 238 to the brain (not shown), where such pulses are perceived as sound.

FIG. 1A provides a schematic of an exemplary conceptual sleep apnea system 1991. Here, this exemplary sleep apnea system utilizes a microphone 12 (represented conceptually) to capture a person's breathing or otherwise the sounds made by a person while sleeping. The microphone transduces the captured sound into an electrical signal which is provided via electrical leads 198 to the main unit 197, which includes a processor unit that can evaluate the signal from leads 198 or, in another arrangement, unit 197 is configured to provide that signal to a remote processing location via the Internet or the like, where the signal was evaluated. Upon an evaluation that an action should be taken or otherwise can be utilitarian taken by the sleep apnea system 1991, the unit 197 activates to implement sleep apnea countermeasures, which countermeasures are conducted by a hose 1902 sleep apnea mask 195. By way of example only and not by way of limitation, pressure variations can be used to treat the sleep apnea upon an indication of such an occurrence.

In an exemplary embodiment, the tinnitus mitigation methods and devices detailed herein can be combined with the sleep apnea system to mitigate tinnitus while treating sleep apnea.

FIGS. 1B and 1C provide another exemplary schematic of another exemplary conceptual sleep apnea system 1992. Here, the sleep apnea system is different from that of FIG. 1A in that electrodes 194 (which can be implanted in some embodiments) are utilized to provide stimulation to the human who is experiencing a sleep apnea scenario. FIG. 1B illustrates an external unit, and FIG. 1C illustrates the external unit 120 and an implanted unit 110 in signal communication via an inductance coil 707 of the external unit and a corresponding implanted inductance coil (not shown) of the implanted unit, according to which the teachings herein can be applicable. Implanted unit 110, can be configured for implantation in a recipient, in a location that permits it to modulate nerves of the recipient 100 via electrodes 194. In treating sleep apnea, implant unit 110 and/or the electrodes thereof can be located on a genioglossus muscle of a patient. Such a location is suitable for modulation of the hypoglossal nerve, branches of which run inside the genioglossus muscle.

External unit 120 can be configured for location external to a patient, either directly contacting, or close to the skin of the recipient. External unit 120 may be configured to be affixed to the patient, for example, by adhering to the skin of the patient, or through a band or other device configured to hold external unit 120 in place. Adherence to the skin of external unit 120 may occur such that it is in the vicinity of the location of implant unit 110 so that, for example, the external unit 120 can be in signal communication with the implant unit 110 as conceptually shown, which communication can be via an inductive link or an RF link or any link that can enable treatment of sleep apnea using the implant unit and the external unit. External unit 120 can include a processor unit 198 that is configured to control the stimulation executed by the implant unit 110. In this regard, processor unit 198 can be in signal communication with microphone 12, via electrical leads, such as in an arrangement where the external unit 120 is a modularized component, or via a wireless system, such as conceptually represented in FIG. 1C.

A common feature of both of these sleep apnea treatment systems is the utilization of the microphone to capture sound, and the utilization of that captured sound to implement one or more features of the sleep apnea system. In some embodiments, the teachings herein are used with the sleep apnea device just detailed.

Returning back to hearing prosthesis devices, in individuals with a hearing deficiency who may have some residual hearing, an implant or hearing instrument may improve that individual's ability to perceive sound. Multimodal prosthesis 200 may comprise an external component assembly 242 which is directly or indirectly attached to the body of the recipient, and an internal component assembly 244 which is temporarily or permanently implanted in the recipient. External component assembly 242 is also shown in FIG. 1D. In embodiments of the present invention, components in the external assembly 242 may be included as part of the implanted assembly 244, and vice versa. Also, embodiments of the present invention may be used with implanted multimodal system 200 which are fully implanted. Embodiments of the teachings herein include utilizing such in the device of FIG. 1D or FIG. 2 detailed below.

External assembly 242 typically comprises a sound transducer 220 for detecting sound, and for generating an electrical audio signal, typically an analog audio signal. In this illustrative arrangement, sound transducer 220 is a microphone. In alternative arrangements, sound transducer 220 can be any device now or later developed that can detect sound and generate electrical signals representative of such sound. An exemplary alternate location of sound transducer 220 will be detailed below.

External assembly 242 also comprises a signal processing unit, a power source (not shown), and an external transmitter unit. External transmitter unit 206 comprises an external coil 208 and, preferably, a magnet (not shown) secured directly or indirectly to the external coil 208. The signal processing unit processes the output of microphone 220 that is positioned, in the depicted arrangement, by outer ear 201 of the recipient. The signal processing unit generates coded signals using a signal processing apparatus (sometimes referred to herein as a sound processing apparatus), which can be circuitry (often a chip) configured to process received signals—because element 2130 contains this circuitry, the entire component 2130 is often called a sound processing unit or a signal processing unit. These coded signals can be referred to herein as a stimulation data signals, which are provided to external transmitter unit 206 via a cable 247 and to the receiver in the ear 250 via cable 252. In this exemplary arrangement of FIG. 1D, cable 247 includes connector jack 221 which is bayonet fitted into receptacle 219 of the signal processing unit 230 (an opening is present in the dorsal spine, which receives the bayonet connector, in which includes electrical contacts to place the external transmitter unit into signal communication with the signal processor 230). It is also noted that in alternative arrangements, the external transmitter unit is hardwired to the signal processor subassembly 230. That is, cable 247 is in signal communication via hardwiring, with the signal processor subassembly. (The device of course could be disassembled, but that is different than the arrangement shown in FIG. 1D that utilizes the bayonet connector.) FIG. 1E provides additional details of an exemplary receiver 250. The overall component containing the signal processing unit is, in this illustration, constructed and arranged so that it can fit behind outer ear 201 in a BTE (behind-the-ear) configuration, but may also be worn on different parts of the recipient's body or clothing.

In some arrangements, the signal processor (also referred to as the sound processor) may produce electrical stimulations alone, without generation of any acoustic stimulation beyond those that naturally enter the ear. While in still further arrangements, two signal processors may be used. One signal processor is used for generating electrical stimulations in conjunction with a second speech processor used for producing acoustic stimulations.

As shown in FIGS. 1D and 1E, a receiver in the ear 250 is connected to the spine of the BTE (a general term used to describe the part to which the battery 270 attaches, which contains the signal (sound) processor and supports various components, such as the microphone—more on this below) through cable 252 (and thus connected to the sound processor/signal processor thereby). Receiver in the ear 250 (as distinguished from a simple receiver—the body of the receiver in the ear 250 supports a receiver—more on this in a moment) includes a housing 256, which may be a molding shaped to the recipient. Inside receiver in the ear 250 there is provided a capacitor 258, receiver 260 and protector 262. Also, there may a vent shaft 264 (in some arrangements, this vent shaft is not included). Receiver in the ear may be an in-the-ear (ITE) or completely-in-canal (CIC) configuration.

In an exemplary arrangement, sound transducer 220 can be located on element 250 (e.g., opposite element 262, as seen for example in FIG. 1F), so that the natural wonders of the human ear can be utilized to funnel sound in a more natural manner to the sound transducer. In an exemplary arrangement, sound transducer 242 is in signal communication with remainder of the BTE device via cable 252, as is schematically depicted in FIG. 1F via the sub cable extending from sound transducer 242 to cable 252.

Also, FIG. 1D shows a removable power component 270 (sometimes battery back, or battery for short) directly attached to the base of the body/spine 230 of the BTE device. As seen, the BTE device in some embodiments includes control buttons 274. The BTE device may have an indicator light 276 on the earhook to indicate operational status of signal processor. Examples of status indications include a flicker when receiving incoming sounds, low rate flashing when power source is low or high rate flashing for other problems.

Returning to FIG. 1, internal components 244 comprise an internal receiver unit 212, a stimulator unit 226 and an electrode assembly 218. Internal receiver unit 212 comprises an internal transcutaneous transfer coil (not shown), and preferably, a magnet (also not shown) fixed relative to the internal coil. Internal receiver unit 212 and stimulator unit 226 are hermetically sealed within a biocompatible housing. The internal coil receives power and data from external coil 208, as noted above. A cable or lead of electrode assembly 218 extends from stimulator unit 226 to cochlea 232 and terminates in an array 234 of electrodes 236. Electrical signals generated by stimulator unit 226 are applied by electrodes 236 to cochlea 232, thereby stimulating the auditory nerve 238.

In one arrangement, external coil 208 transmits electrical signals to the internal coil via a radio frequency (RF) link. The internal coil is typically a wire antenna coil comprised of at least one and preferably multiple turns of electrically insulated single-strand or multi-strand platinum or gold wire. The electrical insulation of the internal coil is provided by a flexible silicone molding (not shown). In use, internal receiver unit 212 may be positioned in a recess of the temporal bone adjacent to outer ear 201 of the recipient.

As shown in FIG. 1, multimodal system 200 is further configured to interoperate with a user interface 280 and an external processor 282 such as a personal computer, workstation, or the like, implementing, for example, a hearing implant fitting system. Although a cable 284 is shown in FIG. 1A between implant 200 and interface 280, a wireless RF communication may also be used along with remote 286.

While FIG. 1 shows a multimodal implant in the ipsilateral ear, in other arrangements, the multimodal implant may provide stimulation to both ears. For example, a signal processor may provide electrical stimulation to one ear and provide acoustical stimulation in the other ear.

With the above as a primer, arrangements are directed to non-multimodal hearing aids utilizing behind the ear devices (traditional acoustic hearing aids using the teachings herein), and non-multimodal external components of cochlear implants utilizing behind the ear devices (traditional external components of such, embodied in a BTE apparatus, utilizing the teachings herein), and some embodiments are directed to multi-modal arrangements utilizing the teachings herein. Still, as will be detailed, embodiments are also directed to multimodal hearing devices.

That is, while the teachings associated with FIGS. 1, 1D, and 2 (discussed below) disclose an external device with an output that is provided external to the recipient (a receiver/speaker) that is in the form of a conventional hearing prosthesis, the disclosure of such and any teachings herein relating to such also correspond to a disclosure of an external device where the output is a bone conduction vibrator. By way of example, a passive transcutaneous bone conduction device, where the conceptual functionality of element 250 (more on this below) could instead be located at a location in back of the ear in a manner concomitant with such (this being a conceptual representation of the placement of the output device), held by magnets to the head of the recipient as conventional in the art. Also by way of example, the external device can be a percutaneous bone conduction device. These components need not be part of a multimodal hearing prosthesis, but could be standalone devices. Moreover, while the teachings associated with FIGS. 1 and 1D are directed towards a cochlear implant, disclosure of such and any teachings herein relating to such also correspond to a disclosure of an implantable/implanted device where the output is a bone conduction vibrator (such as in the case of an active transcutaneous bone conduction device, where the device of FIG. 1D would be readily understood as an external component of such (with or without the conventional hearing aid functionality) or a middle ear actuator (again, where the device of FIG. 1D would be readily understood as an external component of such) or a direct acoustic cochlear stimulator actuator (again, FIG. 1D being a representative external component of such), or any other implanted mechanical device that imparts mechanical energy to tissue of the recipient. Put another way, the disclosure of the output devices relating to the external component vis-à-vis the receiver also corresponds to a disclosure of an alternate embodiment where the output device is a vibrator of a bone conduction device by way of example. Also, the disclosure of the output device relating to the implanted component vis-à-vis the cochlear implant electrode array also corresponds to a disclosure of an alternate embodiment where the output device is a vibrator of a bone conduction device or the actuator of a middle ear implant or the actuator of a direct acoustic cochlear stimulator, by way of example.

FIG. 2 depicts an exemplary BTE device 342 according to an exemplary arrangement. As seen, BTE device 342 includes element 330, which functionally and structurally can, in some arrangements, correspond to element 230 above, with exceptions according to the teachings herein, and thus corresponds to the spine of the BTE device. However, hereinafter, element 330 will be referred to by its more generic name as the signal processor sub-assembly, or sometimes the electronics component of the BTE device, or sometimes, for short, the signal processor, or sound processor subassembly, or sound processor for short (but that is distinguished from the processor therein, which processes sound/signals, and are also referred to as a sound processor or signal processor—this is the pure electronics portion of the overall signal processor subassembly, the latter having a housing and supporting other components), in some instances. As can be seen, attached thereto is element 270 which is thus a power component of the BTE device, which in some instances herein will be referred to as the battery sub-assembly, or the battery for short. The battery sub-assembly 270 is removably attached to the sound processor sub-assembly 330 via, for example, a bayonet connector, the details of which will be described below.

In an exemplary arrangement, BTE device 342 is a conventional hearing aid apparatus. In the ear component 250 can correspond to any of those detailed herein and/or variations thereof. Simply put, the behind the ear device 342 is a conventional hearing aid configured for only external use. It is not an implantable component and does not include implantable components, and is not configured to electromagnetically communicate with an implantable component. Embodiments include one or more or all of the teachings herein embodied in the device of FIG. 2. Also, it is noted that while the receiver/speaker of the device of FIG. 2 is in an in the ear component 250, in other embodiments, the speaker can be adjacent the ear, above the ear, etc. Also, it is noted that earbuds or a headset can be utilized in some arrangements, which can be connected to an MP3 player or to a smart phone, etc. Moreover, a totally in the ear device can be used with one or more of the teachings herein, wherein the totally in the ear device has one or more or all of the features of the conventional hearing aid devices herein and/or other prostheses detailed herein.

It is noted that the teachings detailed herein and/or variations thereof can be utilized with a non-totally implantable prosthesis. That is, in some arrangements, the cochlear implant 200 is a traditional hearing prosthesis. The teachings herein can also be implemented in and in some arrangements are so implemented with respect to other types of prostheses, such as middle ear implants, active transcutaneous bone conduction devices, passive transcutaneous bone conduction deices, percutaneous bone conduction devices, and traditional acoustic hearing aids, alone or in combination with each outer (and/or with the cochlear implant), the combination achieving the bimodal prosthesis. Also, in some embodiments, the teachings detailed herein and/or variations thereof include the teachings herein utilized in totally implantable prostheses, such as those that are totally implantable middle ear implants, active transcutaneous bone conduction devices, alone or in combination with each outer (and/or with the cochlear implant), the combination achieving the multimodal prosthesis.

To be clear, the prostheses herein can include any one or more of an acoustic hearing aid, a percutaneous bone conduction device, a passive transcutaneous bone conduction device, an active transcutaneous bone conduction device, a middle ear implant, a DACS, a cochlear implant, a dental bone conduction device, etc. Thus, any disclosure of one corresponds to a disclosure of any of the others herein and thus a disclosure of using the teachings associated with one with the others unless otherwise noted and unless the art enables such.

FIG. 2A depicts an exemplary system 2110 according to an exemplary arrangement, including device 100, which can be a hearing prosthesis, or a tinnitus mitigation device such as that disclosed in FIG. 2C below, or any device configured to provide stimulation to a recipient that can mitigate tinnitus. In an exemplary arrangement, device 100 corresponds to BTE device 342 or to the prosthesis of FIG. 1, or to the device of FIG. 2C below, etc. Also seen in the system is a portable body carried device (e.g. a portable handheld device as seen in FIG. 2A, a watch, a pocket device, etc.) 2140 in the form of a mobile computer (e.g., a smart phone) having a display 2142. The system includes a wireless link 2130 between the portable handheld device 2140 and the hearing prosthesis 100 (often, 100 is referred to as a hearing prosthesis, and such reference corresponds to a disclosure of an alternate embodiment where such is one of the other devices herein). In an embodiment, the prosthesis 100 is a totally external prosthesis, such as the device of FIG. 2, and in other embodiments, it includes an implanted portion implanted in recipient 99 (as represented functionally by the dashed lines of box 100 in FIG. 2A).

In an exemplary arrangement, the system 2110 is configured such that the hearing prosthesis 100 (which in other embodiments, as noted above, can be a tinnitus mitigation device, such as a masker, or one or more ear buds, or the device 342 of FIG. 2, etc.) and the portable handheld device 2140 have a symbiotic relationship. In an exemplary arrangement, the symbiotic relationship is the ability to display data relating to, and, in at least some instances, the ability to control, one or more functionalities of the hearing prosthesis 100. In an exemplary arrangement, this can be achieved via the ability of the handheld device 2140 to receive data from the hearing prosthesis 100 via the wireless link 2130 (although in other exemplary arrangements, other types of links, such as by way of example, a wired link, can be utilized—concomitant with one or more ear buds connected to the device 2140). As will also be detailed below, this can be achieved via communication with a geographically remote device in communication with the hearing prosthesis 100 and/or the portable handheld device 2140 via link, such as by way of example only and not by way of limitation, an Internet connection or a cell phone connection. In some such exemplary arrangements, the system 2110 can further include the geographically remote apparatus as well. Again, additional examples of this will be described in greater detail below.

As noted above, in an exemplary arrangement, the portable handheld device 2140 comprises a mobile computer and a display 2142. In an exemplary arrangement, the display 2142 is a touchscreen display. In an exemplary arrangement, the portable handheld device 2140 also has the functionality of a portable cellular telephone. In this regard, device 2140 can be, by way of example only and not by way of limitation, a smart phone as that phrase is utilized generically. That is, in an exemplary arrangement, portable handheld device 2140 comprises a smart phone, again as that term is utilized generically.

It is noted that in some other arrangements, the device 2140 need not be a computer device, etc. It can be a lower tech recorder, or any device that can enable the teachings herein.

In an exemplary arrangement, device 2140 can execute or otherwise be utilized for processing purposes associated with the prosthesis 100, such as processing captured sound, and the processed results are then conveyed to the prosthesis via link 2130, where the prosthesis uses those results to evoke a hearing percept.

The phrase “mobile computer” entails a device configured to enable human-computer interaction, where the computer is expected to be transported away from a stationary location during normal use. Again, in an exemplary arrangement, the portable handheld device 2140 is a smart phone as that term is generically utilized. However, in other arrangements, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some arrangements. (As will be detailed below, in some instances, device 2140 is not a mobile computer, but instead a remote device (remote from the hearing prosthesis 100. Some of these arrangements will be described below).)

In an exemplary arrangement, the portable handheld device 2140 is configured to receive data from a hearing prosthesis and present an interface display on the display from among a plurality of different interface displays based on the received data. Exemplary arrangements will sometimes be described in terms of data received from the hearing prosthesis 100. However, it is noted that any disclosure that is also applicable to data sent to the hearing prosthesis from the handheld device 2140 is also encompassed by such disclosure, unless otherwise specified or otherwise incompatible with the pertinent technology (and vice versa).

It is noted that in some arrangements, the system 2110 is configured such that prosthesis 100 and the portable device 2140 have a relationship. By way of example only and not by way of limitation, in an exemplary arrangement, the relationship is the ability of the device 2140 to serve as a remote microphone for the prosthesis 100 via the wireless link 2130. Thus, device 2140 can be a remote mic. That said, in an alternate arrangement, the device 2140 is a stand-alone recording/sound capture device.

It is noted that in at least some exemplary arrangements, the device 2140 corresponds to an Apple Watch™ Series 1 or Series 2, as is available in the United States of America for commercial purchase as of Jun. 6, 2020. In an exemplary arrangement, the device 2140 corresponds to a Samsung Galaxy Gear™ Gear 2, as is available in the United States of America for commercial purchase as of Jul. 20, 2020. The device is programmed and configured to communicate with the prosthesis and/or to function to enable the teachings detailed herein.

In an arrangement, a telecommunication infrastructure can be in communication with the hearing prosthesis 100 and/or the device 2140. By way of example only and not by way of limitation, a telecoil 2149 or some other communication system (Bluetooth, etc.) is used to communicate with the prosthesis and/or the remote device. FIG. 2B depicts an exemplary quasi-functional schematic depicting communication between an external communication system 2149 (e.g., a telecoil), and the hearing prosthesis 100 and/or the handheld device 2140 by way of links 2177 and 2179, respectively (note that FIG. 2B depicts two-way communication between the hearing prosthesis 100 and the external audio source 2149, and between the handheld device and the external audio source 2149— in alternate arrangements, the communication is only one way (e.g., from the external audio source 2149 to the respective device)).

FIG. 2C provides an exemplary tinnitus mitigation system. Here, the system is embodied in a self-contained tinnitus mitigation device 2177. This device can correspond to the smart phone 2140 detailed above, or can be a dedicated device specifically designed for tinnitus mitigation. In this regard, tinnitus mitigation device 2177 includes and earbud jack to which is connected one or more earbuds 2155. In an exemplary embodiment, the tinnitus mitigation device 2177 outputs tinnitus masking sounds (which constitutes tinnitus mitigation as utilized herein). In an exemplary embodiment, the tinnitus mitigation device 2177 outputs sound based mitigation that can be utilized to prevent one set of tinnitus in the first place. Tinnitus mitigation device 2177 includes display screen 2133 as can be seen. This can be the screen of a smart phone of an alternative embodiment (in an exemplary embodiment, device 2177 is a smart phone with earbuds, and in other embodiments, there are no earbuds—the speaker is utilized instead), or can be a dedicated screen of a dedicated tinnitus mitigation device 2177. The screen can provide output to the recipient warning him or her to do something different to avoid the onset of tinnitus (or to reduce the likelihood that tinnitus will occur—any disclosure herein of avoiding the onset of tinnitus corresponds to a disclosure of reducing the likelihood that tinnitus will occur, and vice versa, unless otherwise noted). That constitutes tinnitus mitigation/management. Alternatively, and/or in addition to this, the earbuds or speaker 2166 can output to the recipient output warning him or her to do something different to avoid the onset of tinnitus/reduce the likelihood of the onset of tinnitus. Note that this is not sound based mitigation even though sound is utilized, as that phrase is utilized. Still with respect to speaker 2166, this can also be used to provide sound based mitigation. The speaker can correspond to the speaker of a smart phone in some embodiments. Also as can be seen, there is a microphone 2188. In an exemplary embodiment, this can receive input from the user thereof and/or can receive input indicative of a portion of the ambient environment of the device, such as the audio environment. As we detail below, in an embodiment there are devices and systems that are configured to log ambient audio environment and were to capture the ambient audio environment and evaluate such to determine whether or not a tinnitus event is statistically likely to occur and/or whether or not such is occurring and/or to determine a characterization of a tinnitus event that is occurring or is likely to occur.

Further as can be seen, tinnitus mitigation device 2177 includes a transceiver 2144 and/or a transmitter and/or a receiver that can communicate with another device, such as a remote device or a server that can be utilized to perform analysis and/or processing as will be detailed below. In an exemplary embodiment, the mitigation device can communicate with a remote device utilizing Bluetooth and/or utilizing cellular technology, etc. Alternatively, and/or in addition to this, tinnitus mitigation device 2177 can utilize wired communications to communicate with remote devices etc. It is noted that tinnitus mitigation device 2177 can communicate with a cell phone or a smart phone or with a hearing prosthesis, etc. Also, device 2144 can be utilized to communicate with a device that provides stimulation to a person to mitigate tinnitus, such as by way of example, a wireless earbud system, or to the behind the ear device of FIG. 2, or any other prosthesis that can enable the teachings detailed herein with a modicum of modification, etc. In an exemplary embodiment, tinnitus mitigation device includes electronic circuitry and logic that can enable one or more all of the method actions detailed herein as will be described in greater detail below.

It is also noted that in another exemplary system, tinnitus mitigation can be achieved via an MP3 player or the like that provides an output signal to microphones and/or to earbuds, etc. In an exemplary embodiment, certain sounds or recordings or the like can be stored in the MP3 player and utilized for tinnitus mitigation, when such is activated upon a determination that tinnitus is occurring and/or that a tinnitus event is likely to occur. That said, in an exemplary embodiment, other consumer electronic devices, such as a computer or a tape player even can be utilized for tinnitus mitigation. In an exemplary embodiment, via the Internet for example, sounds for tinnitus mitigation can be accessed in an automated or manual fashion. Any device, system, or method that can enable tinnitus mitigation can be utilized in at least some exemplary embodiments

At least some exemplary embodiments according to the teachings detailed herein utilize advanced machine learning/processing techniques, which are able to be trained or otherwise are trained to detect higher order, and/or non-linear statistical properties of input, which input can be any of the inputs detailed herein (more on this below). An exemplary input processing technique is the so called deep neural network (DNN). At least some exemplary embodiments utilize a DNN (or any other advanced learning signal processing technique) to process one or more inputs (again, as detailed by way of example herein). At least some exemplary embodiments entail training input processing algorithms to process one or more inputs. That is, some exemplary methods utilize learning algorithms or regimes or systems such as DNNs or any other system that can have utilitarian value where that would otherwise enable the teachings detailed herein to analyze inputs. It is noted that in many instances herein, the input will be captured sound in an ambient environment of a microphone. It is noted that the teachings detailed herein can also be applicable to captured light. In this regard, the teachings detailed herein can be utilized to analyze or otherwise process other inputs, such as time of day, data indicative of a physiological feature of user, etc. (more on this below).

A “neural network” is a specific type of machine learning system. Any disclosure herein of the species “neural network” constitutes a disclosure of the genus of a “machine learning system.” Trained neural networks are used in some embodiments. While embodiments herein focus on the species of a neural network, it is noted that other embodiments can utilize other species of machine learning systems accordingly, any disclosure herein of a neural network constitutes a disclosure of any other species of machine learning system that can enable the teachings detailed herein and variations thereof. To be clear, at least some embodiments according to the teachings detailed herein are embodiments that have the ability to learn without being explicitly programmed. Accordingly, with respect to some embodiments, any disclosure herein of a device or system constitutes a disclosure of a device and/or system that has the ability to learn without being explicitly programmed, and any disclosure of a method constitutes actions that results in learning without being explicitly programmed for such.

Some of the specifics of the DNN utilized in some embodiments will be described below, including some exemplary processes to train such DNN. First, however, some of the exemplary methods of utilizing such a DNN (or any other system having utilitarian value) will be described.

It is noted that in at least some exemplary embodiments, the DNN or the product from machine learning, etc., is utilized to achieve a given functionality as detailed herein. In some instances, for purposes of linguistic economy, there will be disclosure of a device and/or a system that executes an action or the like, and in some instances structure that results in that action or enables the action to be executed. Any method action detailed herein or any functionality detailed herein or any structure that has functionality as disclosed herein corresponds to a disclosure in an alternate embodiment of a DNN or product from machine learning, etc., that when used, results in that functionality, unless otherwise noted or unless the art does not enable such.

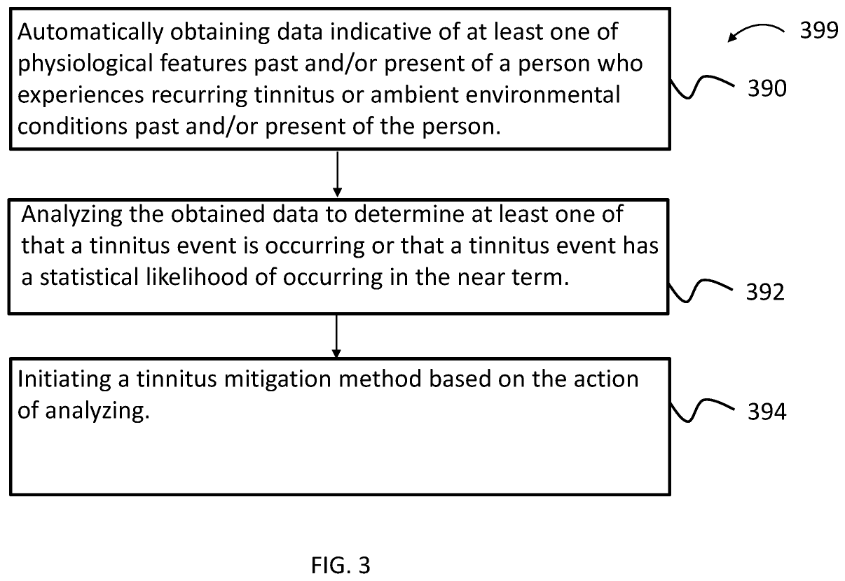

FIG. 3 depicts an exemplary flowchart for an exemplary method, method 399, of utilizing, by way of example, in some embodiments, a product of and/or from machine learning, such as a trained neural network (which includes a neural network that is continuing to be “remedially” trained, in the sense that the network can be used to achieve utilitarian results, but the teachings herein include continuously training a network during use of that network—more on this below) by way of example only and not by way of limitation, according to an exemplary embodiment, while in other embodiments, the method is executed utilizing standard electronics configured to execute the method actions herein. Method 300 includes method action 390, which includes obtaining, and in some embodiments, automatically obtaining, data indicative of at least one of physiological features past and/or present of a person who experiences recurring tinnitus or ambient environmental conditions past and/or present of the person. Also, embodiments include obtaining additional data, such as prosthesis device settings, etc. Additional details of this data will be provided below, but here, it is briefly noted that in at least some exemplary embodiments, the smart phone 2140 and/or the hearing prosthesis 342, or 100, etc., such as that embodied in the embodiment of FIG. 2, or a tinnitus masker apparatus with expanded functionality, such as the ability to receive input and logic circuitry that can evaluate the input (more on this below) or a smart phone-based device utilizing earbuds that provide tinnitus mitigation (again more on this below), or even a desktop or laptop PC, can be utilized in at least some exemplary embodiments to execute method action 390. It is briefly noted that the action of automatically obtaining data is executed when the data obtaining functionality is activated by a person involved in the execution of the method. That is, the mere activation of a data obtaining functionality of a device does not correspond to automatically obtaining—it is what happens after activation of that functionality that corresponds to method action 390.

Method 399 further includes method action 392, which includes analyzing the data obtained in method action 390 to determine at least one of that a tinnitus event is occurring or that a tinnitus event has a statistical likelihood of occurring in the near term. In an exemplary embodiment by way of example only and not by way of limitation, the action of analyzing is executed using the results from machine learning or any other artificial intelligence/machine learning principles that can have utilitarian value and otherwise can enable at least some of the teachings detailed herein. In an exemplary embodiment, method action 392 is executed using a device that includes a product of and/or resulting from machine learning. In an exemplary embodiment, method action 392, as with all method actions herein, can be executed automatically (and in some alternate embodiments, one or more method actions detailed herein can be executed not automatically—any disclosure herein of any method action or functionality corresponds to a disclosure where such is executed automatically, and an alternative embodiment where such is not executed automatically, unless otherwise noted and providing that the art enables such). In an exemplary embodiment, any method action and/or functionality disclosed herein can be performed by a human, and such disclosure of such actions and/or functionality corresponds to an exemplary embodiment of such.

In an exemplary embodiment, the product is a chip that is fabricated based on the results of machine learning. In an exemplary embodiment, the product is a neural network, such as a deep neural network (DNN). The product can be based on or be from a neural network. In an exemplary embodiment, the product is code (such as code loaded into the smartphone 2140, or into the prosthesis 342 (or any prosthesis herein, or any tinnitus masker/tinnitus mitigation device as described herein by way of example). In an exemplary embodiment, the product is a logic circuit that is fabricated based on the results of machine learning. The product can be an ASIC (e.g., an artificial intelligence ASIC). The product can be implemented directly on a silicon structure or the like. Any device, system, and/or method that can enable the results of artificial intelligence to be utilized in accordance with the teachings detailed herein, such as in a hearing prosthesis or a component that is in communication with a hearing prosthesis, can be utilized in at least some exemplary embodiments. Indeed, as will be detailed below, in at least some exemplary embodiments, the teachings detailed herein utilize knowledge/information from an artificial intelligence system or otherwise from a machine learning system.

Exemplary embodiments include utilizing a trained neural network to implement or otherwise execute at least one or more of the method actions detailed herein, and thus embodiments include a trained neural network configured to do so. Exemplary embodiments also utilize the knowledge of a trained neural network/the information obtained from the implementation of a trained neural network to implement or otherwise execute at least one or more of the method actions detailed herein, and accordingly, embodiments include devices, systems, and/or methods that are configured to utilize such knowledge. In some embodiments, these devices can be processors and/or chips that are configured utilizing the knowledge. In some embodiments, the devices and systems herein include devices that include knowledge imprinted or otherwise taught to a neural network. The teachings detailed herein include utilizing machine learning methodologies and the like to establish tinnitus mitigation systems and/or devices and/or sensory prosthetic devices or supplemental components utilized with sensory prostatic devices or with tinnitus mitigation devices (e.g., a smart phone) and/or tinnitus mitigation devices embodied in consumer electronic devices (e.g., a smartphone with earbud(s) to provide masking, etc.) to identify when and/or what type of tinnitus mitigation is utilitarian and to engage/enables such.

As noted above, method action 392 can entail analyzing, including processing, the data utilizing a product of machine learning, such as the results of the utilization of a DNN, a machine learning algorithm or system, or any artificial intelligence system that can be utilized to enable the teachings detailed herein. This as contrasted from, for example, processing the data utilizing general code or utilizing code that does not from a machine learning algorithm or utilizing a non AI based/resulting chip, etc. Although it is noted that in other embodiments, such is utilized as well, such as, for example, method 392, which is executed only by way of example via a DNN, and can be executed utilizing a product that is not of machine learning. In an exemplary embodiment, a hearing prosthesis and/or the smart phone or other personal electronics device and/or a tinnitus mitigation device, etc., processes a signal from a microphone and subsequently provides the results of that processing to a control device that, depending on the results of the processing (a tinnitus event is statistically likely to occur in the near-term or not), activates a tinnitus mitigation method (more on this in a moment).

According to at least some exemplary embodiments, a feedback loop is provided that receives data associated with tinnitus events. The trained neural network (or, neural network in training) is part of this feedback loop in some embodiments, and utilizes the feedback to learn how to better mitigate tinnitus.

Again, in an exemplary embodiment, the machine learning can be a DNN, and the product can correspond to a trained DNN and/or can be a product based on or from the DNN (more on this below).

FIG. 3 further includes method action 394, which includes initiating a tinnitus mitigation method based on the action of analyzing in method action 392 (here, the action of analyzing has determined that there is a statistical likelihood of occurrence of a tinnitus event in the near-term). In an exemplary embodiment where method 399 is executed utilizing a hearing prosthesis and/or a tinnitus masker and/or a dedicated tinnitus mitigation device or utilizing equipment that can be used for tinnitus mitigation, (e.g., smart phone or a computer, etc.), the method includes providing a sound to the person that is the subject of the method that will mask the one coming tinnitus. In an exemplary embodiment, method action 390, 392, and/or method action 394 can be executed by separate device(s), such as by way of example only and not by way of limitation, device 2140 or 2177, etc., and such devices can be utilized to execute method action 392 and the hearing prosthesis 100/342 can be utilized to execute method action 394.

In an exemplary embodiment, tinnitus mitigation can include providing a sound that masks the tinnitus, providing a sound that reduces the likelihood of the tinnitus event from occurring in the first instance (which includes preventing such) and/or instructing the person suffering from tinnitus to take certain actions that reduces the likelihood of the tinnitus event from occurring in the first instance (e.g., shutting down a sound source, having a person exit the environment, having a person utilize earplugs, having a person move to elevate heart rate, having a person drink a cup of coffee or eat a salty food, etc.).

In an exemplary embodiment, based on the results of method action 392, an indication can be provided to a person who suffers from tinnitus to utilize the tinnitus mitigation device or otherwise take any of the aforementioned actions or other actions noted above, thus executing method action 394.

Indeed, in an exemplary embodiment, embodiments include any variations of the devices and systems detailed herein that are configured to control certain aspects of an ambient environment of a person. By way of example only and not by way of limitation, with respect to an infrastructure where there are such control regimes in place, the device can instruct a building control system to dim lights or to brighten lights or to shut off certain lights. The devices and systems can instruct or otherwise control other devices, such as televisions and/or radios, to automatically engage in certain actions (increased volume, decreased volume, change channel, play a certain sound, or play certain background noises, etc.). The devices and systems can activate certain devices, such as TVs or radios or shut such devices down. All of this based on the results of method action 392. Of course, in some such embodiments, the infrastructure would be relatively intense as compared to simply issuing an instruction or recommendation to turn off the television or the like, but as of the filing of this application, the technology exists to integrate any of the teachings detailed herein with an overall control regime that can control and ambient environment of a person.

Still further, with respect to the action of obtaining data of method action 390, the Internet of things can be utilized in some exemplary embodiments. In an exemplary embodiment, the microphones of a computer or the microphones of a telephone, etc., can be utilized to capture and auditory environment. The Alexa device can be utilized to capture sound and/or to implement method action 394. All of these can be implemented in at least some exemplary embodiments utilizing wireless technology that is readily available, and accordingly, at least some exemplary embodiments include utilizing such wireless technology to achieve any one or more of the above-noted actions and/or to integrate any of the devices detailed herein with devices in an environment that can be controlled in a method of mitigating tinnitus.

In an exemplary embodiment, a remote device, such as a remote server, can be utilized to execute method action 392, where, for example, method action 390 is executed by a component that is in the possession of the person who suffers from tinnitus (e.g., a hearing prosthesis and/or the smart device 2140, or any other device that can enable method action 390), and this component then provides data to a remote server via the Internet or via Bluetooth or via any other data communication arrangement, such as via cellular system, etc., and the remote server executes or otherwise has access to a device configured to execute method action 392, and then method action 392 is executed. The remote server then communicates results of method action 392 back to the person who is afflicted by tinnitus (and/or to a device in the possession of the person, whether that is the same device or another device), and method action 394 is initiated, whether that is initiated automatically, or manually by the person, by any device that can enable tinnitus mitigation according to the teachings detailed herein and/or variations thereof.

In at least some exemplary embodiments, consistent with the teachings detailed above, in an exemplary embodiment, all of the actions associated with method 399 are executed by a self-contained body worn and/or body carried sensory prosthesis or other prosthesis or other body carried device that can enable tinnitus mitigation or otherwise can be used in conjunction with such a method, and/or as part of a method (e.g., smartphone), while in other embodiments, such as where processing power is constrained, some of the actions are executed by a device that is separate from the self-contained body worn sensory prosthesis and/or other devices in the possession of the user and/or by a remote devices, and the results of those actions are communicated to the sensory prosthesis and/or the tinnitus mitigation device so that tinnitus mitigation can be executed.

As noted, method 399 is executed in association with a person who experiences recurring tinnitus. This does not mean the person occasionally experiences tinnitus, as do most people. This means that the person has a sufficient problem with tinnitus that he or she seeks to utilize the method in the first instance. In an exemplary embodiment, such a person is a person who is medically diagnosed as having tinnitus.

With respect to the feature of a statistical likelihood of a tinnitus event occurring in the near-term, this means something more than the person experiences recurring tinnitus, such as an event that occurs every day or every few days or multiple times a day based on statistical past experience. Put another way, death is an experience that occurs in the long run, and it occurs to everyone. It is the short run about which one is concerned. Sleep is another experience that would occur in the long run, and it also occurs to everyone at some point. By rough analogy, this is predicting something more specific or probable than that which will eventually occur if given enough time.

Another analogy could be forecasting earthquakes. As of this writing, there are some correlations that indicate that an earthquake sometimes happens, but those indications do not correspond to a statistical likelihood of such. The People's Republic of China (or an entity associated therewith) presented a forecast that was ultimately accurate years ago with respect to an earthquake. The fact that on rare occasions correlations result in the occurrence of a forecasted event does not mean that there is a statistical likelihood of such occurrence, or that that is predictive. Such occurrences do not correspond to predictive prowess or statistical likelihood. To be clear, these rare occurrences are more than the broken clock axiom (it is correct twice a day), and there can be utility to such forecasts, but they are not statistically likely or predictive. Conversely, a statistical likelihood does not mean that it is always the case, 100% of the time, that a given set of circumstances corresponds to an event. By rough analogy, if it is raining, there is a statistical likelihood that people driving on a highway are utilizing their windshield wipers. Rain might be light enough that people are not using the windshield wipers, and some cars, such as mid-90s Corvettes, have windshield angles that at a certain speed, the rain will actually be blown off the windshield, and some drivers may be too lazy to put the wipers on, and some cars may not have wipers that work. But still, statistically speaking, a given car on a highway will have windshield wipers that are on.

Note also that this can be subjective to an individual person. For example, the statistically likelihood can be for an individual, as opposed to a group/population, even within a population of tinnitus suffers/people how experience recurring tinnitus.

In an exemplary embodiment, instead of a near term qualifier, method action 392 is such that a determination that there is a statistical likelihood of the event occurring in less than or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 85, 90, 120, 150, 180 seconds 3.5, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 85, 90, 120, 150, or 180 minutes, or any value or range of values therebetween in 1 second increments (e.g., 4 minutes and 10 seconds, 123 minutes, 33 to 77 minutes, etc.). It is noted that the concept of “near term” encompasses at least some of the quantities just detailed in at least some embodiments.

In an exemplary embodiment, method actions 390, 392, and/or 394 are executed automatically, such as can be the case under the control of a controller that corresponds to a processor or chip or some other logic circuitry that is configured utilizing standard practices that can enable such. By way of example only and not by way of limitation, in an exemplary embodiment, the activation and engagement of the tinnitus mitigation can be executed utilizing any device, system, and/or method that can enable such. In an exemplary embodiment, the control unit(s) of the various prostheses detailed herein and/or the logic circuitry thereof can be modified to initiate the execution and/or execute any one or more of these method actions and/or to have these functionalities. In an exemplary embodiment, an app or the like can be loaded onto a smart phone or the like. A personal computer can be utilized to implement some or more of the method actions detailed herein in an automated fashion.

To be clear, in at least some exemplary scenarios of tinnitus, it is difficult or otherwise hard for a person to learn and understand their tinnitus patterns. Briefly, the machine learning herein can be used to develop a model of the tinnitus patterns of a given person. In at least some exemplary embodiments of the teachings detailed herein, such as those that are implemented in the automated fashions, the systems detailed herein can be utilitarian in this regard. In at least some exemplary embodiments, a system that manages a person's tinnitus automatically can enable a person to not worry about his or her tinnitus and/or worry much less about it or otherwise spend less time dealing with his or her tinnitus. At least some exemplary embodiments permit the tinnitus afflicted person to avail himself or herself to tinnitus mitigation features without the need to consciously interact with an external device(s), an App, and/or adjust setting(s) on and off manually, of a tinnitus mitigation device/device being utilized as such. In this regard, there is utilitarian value with respect to a device that operates in a manner that is not necessarily recognize or otherwise activates and/or deactivates in a manner that is not apparent to the user. Indeed, in an exemplary embodiment, the teachings detailed herein can include a device and/or system that diverts the individual's attention, hence reducing the individual's anxiety of having a concern of not being able to hear things coming up because of the unexpected buzzing/ringing in the ear. In an exemplary embodiment, the diversion of attention can correspond to a tinnitus mitigation function.

In an exemplary embodiment, the action of analyzing (method action 392) results in a determination of the statistical likelihood that a tinnitus event will occur in the near term. This as opposed to a determination of a statistical likelihood that a tinnitus event will not occur, which be the case in some exemplary scenarios—indeed, in at least some exemplary scenarios, that will be the bulk of the results of method action 392, at least for people who do not suffer from tinnitus 24/7—it is briefly noted that the teachings detailed herein include determining the statistical likelihood that a tinnitus event will occur in the near term and/or also determining the statistical likelihood that a tinnitus event will not occur in the near term, and with respect to the latter, the mitigation is not implemented.

In at least some exemplary scenarios of the method 399, the tinnitus event has not yet occurred. In this regard, method action 392 is a predictive action. That said, in alternative embodiments, the tinnitus event has occurred or otherwise is occurring, and method action 392 is an action of determining in real time or as close to real time as possible that the person at issue is experiencing a tinnitus event. In at least some exemplary embodiments, this can be achieved by the person at issue providing input into a system utilized to implement the method but in other embodiments, this is done without affirmative input from the person, and can thus be done automatically. Indeed, in an exemplary embodiment, or more appropriately, in an exemplary scenario, the person does not recognize that he or she is experiencing a tinnitus event in the short term and such an event still occurs in the short term. Accordingly, in an exemplary embodiment, the teachings detailed herein have utilitarian value with respect to keeping a person who experiences a tinnitus episode from recognizing such. By way of example only and not by way of limitation, in an exemplary embodiment, a tinnitus masking device can be utilized and activated prior to or immediately at the onset of the tinnitus episode (or immediately upon determining that an event is occurring or will occur in accordance with method 399) or an otherwise close temporal proximity thereto, to achieve this utilitarian value.

There are embodiments where the teachings detailed herein are utilized to achieve an adaptive as opposed to a reactive tinnitus mitigation regime. The utilization of the predictive teachings herein enables the proactive actions detailed herein that can prevent the onset of the tinnitus event, or at least prevent the noticeability of such in the first instance. In an exemplary embodiment, the devices and systems disclosed herein enable the tracking over time of a person's tinnitus experiences and correlates such with the various data logged and develops and adapts to changing scenarios to further counter or otherwise manage the tinnitus. In an exemplary embodiment, the devices and/or systems detailed herein enable the tracking of these measures over time and evaluate how the various measurements trend over time to develop a tinnitus management regime.

To be clear, at least some embodiments herein rely on masking, which is something that can enable a recipient to avoid the recognition that tinnitus is coming or is actually happening. Also, teachings herein rely on actions that completely avoid the occurrence of tinnitus in the first instance. Any one or both of these regimes can be utilized in at least some embodiments.

Thus, some embodiments of the teachings detailed herein enable the real-time monitoring to avoid tinnitus in the first instance. Indeed, in an exemplary embodiment, the tinnitus mitigation efforts are initiated before the occurrence of tinnitus.

By way of example, in an exemplary embodiment, there includes alleviating/relieving or otherwise managing tinnitus by implementing a masking output, wherein the masking is initiated and/or truncated without manual and/or affirmative input from the person afflicted with the tinnitus. With respect to truncation, it is noted that for textual economy, any disclosure herein of initiation of tinnitus mitigation efforts also corresponds to an alternate disclosure of halting or otherwise stopping tinnitus mitigation efforts, albeit with any appropriate modifications to the underlying data sets or otherwise underlying evaluations that would be utilitarian to determine when to do so.

In an exemplary embodiment, for a statistically significant population of tinnitus sufferers, at least and/or equal to 20, 25, 30, 35, 40, 45, 50, 55, 60, 65, 70, 75, 80, 85, 90, 95, or 100%, or any value or range of values therebetween in 1% increments of tinnitus episodes that occur are not recognized by given person over Z hours of implementation of the method/use of the devices to implement such, within a given W month period, where Z can be 200, 225, 250, 275, 300, 325, 350, 375, 400, 425, 450, 475, 500, 525, 550, 575, 600, 625, 650, 675, 700, 720, 725, 750, 775, 800, 850, 900, 950, 1000, 1050, or 1100 or more, or any value or range of values in 1 increment, and W can be 1, 1.5, 2, 2.5, 3, 3.5, 4, 4.5, 5, 5.5, 6, 6.5, 7, 7.5, 8, 8.5, 9, 9.5, or 10, or any value or range of values therebetween in 0.25 increments. In an exemplary embodiment, this is the case instead for a subjective person within a given W month period. In an exemplary embodiment, at least 10, 15, 20, 25, 30, 35, 40, 45, 50, 60, 70, 80, 90, or 100 or more, or any value or range of values therebetween in 1 increment episodes are not recognized by the given person within the aforementioned temporal periods.

In at least some exemplary scenarios, method action 392, the action of analyzing, results in a determination of the statistical likelihood that a tinnitus event will occur in the near term, the tinnitus event has not yet occurred, the person does not recognize that the mitigation has begun, and the person does not recognize that he or she is experiencing a tinnitus event in the short term. In an exemplary embodiment, for a statistically significant population of tinnitus sufferers, at least and/or equal to 20, 25, 30, 35, 40, 45, 50, 55, 60, 65, 70, 75, 80, 85, 90, 95, or 100% or any value or range of values therebetween in 1% increments of mitigation actions that occur are not recognized by given person over Z hours of implementation of the method/use of the devices to implement such, within a given W month period. In an exemplary embodiment, this is the case instead for a subjective person within a given W month period. In an exemplary embodiment, at least 10, 15, 20, 25, 30, 35, 40, 45, 50, 60, 70, 80, 90, or 100 or more, or any value or range of values therebetween in 1 increment mitigation actions (which are discrete starting from the initiation to the end of the mitigation action) are not recognized by the given person within the aforementioned temporal periods.

In at least some exemplary embodiments, the data automatically obtained in method action 390 is data indicative of the ambient environmental conditions and does not include physiological features. In an exemplary embodiment, the data automatically obtained is data indicative of the ambient environmental conditions and physiological features.

But again to be clear, while some embodiments include data that is automatically obtained, in other embodiments, the data can be obtained in a non-automated manner. By way of example only and not by way of limitation, the physiological states of the user or otherwise person of interest can be obtained by either automatic measures or by manual/person of interest input. In an exemplary embodiment, the devices, systems, and/or methods herein can be configured to receive audio statements by the person of interest and analyze those statements to determine the physiological state. For example, if the person of interest states out loud that he or she is experiencing tinnitus at a given level let us say from a scale of 1 to 10, and/or at a general frequency classification (predetermined, which could have a given name might frequency A or B or C or the like, or low, low-medium, medium, high, etc.) the system can record that or otherwise receive that statement and analyze that statement accordingly. Also, in at least some embodiments, the characterizations detailed below can also be included (scale of 1 to 10, etc.) as will be described for the below. That said, that can constitute data logging as will be described below. In an exemplary embodiment, the person of interest can input data into the smart phone for example. A user input app can exist that enables the person of interest to put in data relating to his or her physiological conditions, in a predetermined manner, via a touch screen of the smart phone.

It is also noted that in at least some embodiments, the devices and systems enable, and methods also include obtaining device settings or other settings related to a prosthesis or other hearing device or other tinnitus mitigation device that the person of interest might be utilizing.

In an exemplary embodiment, data indicative of the ambient environmental conditions can include data related to sound environments, including speech of the person suffering from tinnitus, speech of others, including speech of others speaking directly to the recipient and/or speech of others that the recipient seeks to understand, the presence of other sounds, such as wind noise, equipment noise, music noise, machine noise (fan, HVAC system), general background noise (radio, television), crowd noise, traffic noise, water noise, typing noise, children noise, etc. Further, ambient environmental conditions can include day or night conditions, light or dark conditions, temperature conditions, humidity conditions, location conditions, activity conditions (e.g., driving, exercising, walking, running, swimming, eating, reading, typing, relatively intensive eye focusing), time of day, time of week, prosthesis device settings (including hearing prosthesis settings). Any ambient environmental condition that has a statistically significant correlation on triggering a tinnitus episode or otherwise is correlated to the subsequent occurrence of such or the present existence of such can be included in at least some exemplary embodiments vis-à-vis obtaining data indicative thereof providing that the art enables such. Additional embodiments can include the utilization of locational conditions, such as whether or not a person is at a beach or near a highway or near an airport, etc. Embodiments can also include the utilization of such conditions as whether or not the person is in a car or in an office building or at home or in a bedroom or outside or in a location that has a high reverberant sound basis or a low reverberant sound basis, etc.

Embodiments include devices and systems that enable, and methods of identifying any of the above providing that the art enables such, in an automatic and/or person imported manner. By way of example only and not by way of limitation, any of the devices disclosed herein in some exemplary embodiments, can determine the speech of the person of interest and segregate that from other speech/speech of others. Such can have utilitarian value with respect to utilizing speech of a person suffering from tinnitus as an indicator or otherwise is a latent variable that tinnitus is occurring and/or the tinnitus is about to occur and/or the characterization of the tinnitus, as will be described in greater detail below.

In an exemplary embodiment, certain background noises that have a particular frequency may trigger or otherwise exasperate tinnitus. In some embodiments, this background noise can be the data that is logged by the system and a correlation between such and the onset of tinnitus or the severity of tinnitus can be established. In some embodiments, the tinnitus mitigation regimes may include detecting such background noises and upon such detection, recommending to the recipient that he or she alleviate that background noise (stop the noise, put in ear plugs) or otherwise leave an area where such noise exists. In some embodiments, such as those that utilize features of hearing prostheses, a sound processor can be utilized to change the frequency of the sound that is being perceived by the recipient so as to reduce the likelihood that the tinnitus event will be triggered and/or reduce the severity of the tinnitus event. More on this below.

Embodiments can take into account that tinnitus can have an impact on speech perception. In some instances, a person's speech can be reflective of his or her speech perception. Indeed, by comparing the speech of others to the speech of a person of interest, or even simply evaluating the speech of the person of interest in isolation, it is possible in at least some embodiments to deduce that the person is experiencing a tinnitus event. That is, by utilizing the speech of a person of interest as a latent variable, the speech of the person can be utilized as a marker or otherwise indicia that a tinnitus event is occurring. Put another way, a person speech would be different if he or she was not experiencing a tinnitus event, at least a severe tinnitus event. Embodiments herein utilize the devices and/or systems that are configured to, and include methods of, detecting incidences of poor speech quality and/or different speech patterns of a person of interest, and utilize such as a marker of tinnitus onset, and trigger an appropriate mitigation strategy in an automated fashion on the identification of such. Speech patterns can also be utilized as a proxy or otherwise as a latent variable of tinnitus/that a tinnitus event is occurring. Embodiments include data logging associated with the speech of the person of interest and correlating various speech patterns/quality of speech to tinnitus events in accordance with the teachings detailed herein.

Corollary to the above is that in at least some exemplary embodiments, the tinnitus management/mitigation techniques disclosed herein can actually increase the understandability of speech. In an exemplary embodiment, there is the analysis and/or measurement of speech production deviance in terms of intelligibility ratings, which can be monitored, and can be used as an indicator as to whether or not the tinnitus mitigation is utilitarian. In any event, such can be utilized as a gauge of utilitarian value of the teachings herein. Accordingly, in at least some exemplary embodiments, and overall speech intelligibility score when a standardized speech intelligibility test is increased by at least 10, 15, 20, 25, 30, 35, or 40% or more relative to that which would be the case in the absence of the teachings detailed herein, at least when a tinnitus episode is occurring or otherwise would have occurred based on the statistical data.

Analyzing the speech of a person who is afflicted with tinnitus and/or speech of others and/or comparing the two and/or otherwise capturing data that can be utilized to do so and/or evaluating intelligibility of speech can be performed utilizing any one or more of the teachings detailed in PCT Application Publication No. WO 2020/021487, published on Jan. 30, 2020, entitled Habilitation and/or Rehabilitation Methods And Systems. Indeed, in an exemplary embodiment, any of the teachings of that patent application publication that are related to identifying the speech of a given person, obtaining data associated with the speech of that person, recording the speech of that person, evaluating speech of a given person or the speech of others, can be utilized in at least some exemplary embodiments as a proxy for whether or not a person is experiencing a tinnitus episode (or will likely experience such), and such can correspond to the data detailed herein, providing that the art enables such. Indeed, any disclosure of that patent application publication of utilizing such as a proxy for evaluating how well a person can hear or otherwise extracting indicia associated with a person's hearing, whether such hearing is natural or resulting from stimulation from an artificial prostheses, correspond to an alternate disclosure herein of a modified method and/or modify device and/or system of doing so to identify tinnitus episodes were evaluated tinnitus feature as opposed to the ability to hear.

Physiological data that is obtained can correspond to cognitive load and/or stress levels, and can also be utilizes a proxy for a tinnitus event occurrence. The various sensors detailed herein can be utilized to determine such and/or deduce that there is a high cognitive load and/or a high stress level of a person of interest, and any device, system, and/or method that can enable the inducement of cognitive load and/or stress levels that can enable such to be utilizes a proxy for tinnitus determination to be utilized in at least some exemplary embodiments. Brain activity can also be used as a data set that can be evaluated to deduce the likelihood that a tinnitus event will occur and/or that such is occurring. Indeed, in at least some exemplary embodiments, any one or more emotional responses can be utilized as a data set.

In some embodiments, the aforementioned data that is utilized as a proxy or otherwise is a latent variable of tinnitus may not be present in all people. Indeed, some people do not get bothered by tinnitus. Accordingly, many of the data sets detailed herein can be subjective to a given person. That said, with respect to big data or otherwise utilizing a statistically significant population to develop the algorithms, there can be utilitarian value with respect to excluding certain people from the population, such as those that do not get bothered by tinnitus.

And what of enablement. By way of example only and not By way of limitation, devices, systems and methods can include global positioning systems that provide indication related to the presence or the location of a given person. Some exemplary embodiments can include global positioning systems that are combined with hearing prostheses and/or tinnitus mitigation devices and/or smart phones, etc. Any combination of such they can enable the teachings detailed herein can be utilized in at least some exemplary embodiments. With respect to sound environments, as will be further detail below, in an exemplary embodiment, the microphone of the hearing prosthesis or of the tinnitus mitigation device and/or of the smart phone or other device, can be utilized to capture ambient sound (ambient to the microphone, and thus includes the sound of the person of interest's voice) and the device can be configured to analyze the captured sound and determine or otherwise classify sound environment. By way of example only and not by way of limitation, sound classification and/or scene classification can be executed utilizing any one or more of the teachings of U.S. Patent Application Publication No. 2017/0359659, entitled advance scene classification for prostheses, by the great legendary innovators in the art that go by the names Alex von Brasch, Stephen Fung, and Kieran Reed, published on Dec. 14, 2017. In an exemplary embodiment, any one or more of the teachings detailed therein can be utilized in any device, system, and/or method disclosed herein in combination thereof, providing that the art enables such. In an exemplary embodiment, the classifications that are enabled by the teachings of the '659 publication can be utilized to identify a sound environment or otherwise provide or otherwise create the data that is obtained in method action 390 and/or utilized in method action 392. In an exemplary embodiment, the device utilized to implement method 399 corresponds to any of the devices detailed in the '659 publication and/or variations thereof, such as hearing prostheses corresponding to an acoustic hearing aid along the lines of the embodiment of FIG. 2 having any one or more or all of the features detailed in the '659 publication combined with one or more or all of the teachings detailed herein.

There can be a device configured to tell time that can be utilized to determine time of day. The devices utilized to implement the teachings herein can include an onboard timer or circuitry configured to keep track of elapsed time, and thus time of day and/or day can be correlated thereto in a manner analogous to that which is the case with respect to the operations of a computer with an onboard clock. That said, in an exemplary embodiment, a communications link can be established with a timekeeping device, such as the atomic clock at the Naval Observatory, via the Internet. That said, temporal features can be obtained utilizing devices, systems and methods that are utilized by smart phones or the like.

Moreover, in an exemplary embodiment, the devices, systems disclosed herein can be configured to, and methods disclosed herein include, receive(ing) data from remote devices, such as from televisions or the like, via wired or wireless communication. By way of example, a television can output a signal that can be received by the acoustic hearing aid or whatever device is being utilized, which signal can indicate an environmental condition. Also by way of example the Internet of things can be utilized to obtain some of the data utilized in method 399 and/or the other methods detailed herein. In an exemplary embodiment, the devices and systems are configured to and methods include communicat(ing) with the Internet of things to obtain the data that is utilized in some embodiments. Still further, light sensors or the like or cameras can be utilized to obtain some data. Image recognition systems can be utilized to obtain data that is utilized in some embodiments. It is also noted that the environmental factors noted above can also be factors that are correlated to the perception of tinnitus by the recipient.

As noted above, some embodiments of method 390 utilize data indicative of physiological features. For example only, can be the results of an EEG monitor, an EKG monitor, body temperature, pulse, brain wave/brain activity data, sleeping/awake conditions and/or drowsiness alertness, eye movement/rate of eye-movement data, blood pressure, etc., or any other physiological condition or data set that can enable the teachings detailed herein or otherwise has a statistically significant relationship to determining the onset of a tinnitus event and/or that a tinnitus event is occurring providing that the art enables such.

It is briefly noted that embodiments can include obtaining data relating to whether or not a person of interest is experiencing a headache and/or migraine, whether or not a person of interest has had enough sleep or little sleep or otherwise obtaining the amount of sleep experienced by the person of interest, hormonal issues of the person of interest, whether or not a person is experiencing dizziness or the like, the type of food and/or the last time or how frequently and/or the time frames the person ate, the types of drinks and/or the last time and/or how frequently and/or the time frames the person hydrated or otherwise drank, whether a person experience nausea and the times associated therewith, etc. Any of the aforementioned data can be utilized in accordance with the teachings detailed herein to develop a method to predict and/or identify the currents of tinnitus and/or to correlate features associated therewith. Any the aforementioned data can correspond to the data of method 390.

Any psychoacoustic data set that can have utilitarian value can be utilized in at least some exemplary embodiments. With respect to enabling the art, by way of example only and not by way of limitation, any one or more of the teachings detailed in PCT Application Publication No. WO 2020/089856, published on May 7, 2020, entitled Physiological Measurement Management Utilization Prostheses Technology and/or Other Technology. Indeed, in an exemplary embodiment, any one or more of the physiological features that are measured as disclosed in the '856 publication are utilized as data for method 399. In an exemplary embodiment, any one or more of the devices, systems, and/or method disclosed in the '856 publication are utilized to obtain the data. In an exemplary embodiment, any one or more of the embodiments disclosed in the 856 publication and/or the devices, systems and/or methods disclosed therein are utilized in combination with any one or more of the devices, systems, and/or method disclosed herein to implement any one or more or all of the devices, systems and methods disclosed herein. In some embodiments, any one or more of the prostheses detailed in the '856 publication are utilized in combination with any one or more the devices herein.

It is briefly noted that in at least some exemplary embodiments, method action 392 is executed without affirmative input from the person that is the subject of the method. That is, in an exemplary embodiment, this is concomitant with the concept of automatically identifying that a tinnitus event is occurring or will occur in the short-term, and such is done without input from the person of interest. That said, it is noted that in some exemplary embodiments, there exists affirmative input from the person of interest. Accordingly, in at least some exemplary embodiments, the devices and systems herein are enabled to permit the person of interest to affirmatively input data indicative that he or she is experiencing tinnitus and/or that he or she believes that he or she is about to experience a tinnitus event within the short-term.

An exemplary embodiment includes an apparatus that comprises a body carried portable device including an input subsystem and an output subsystem, wherein the device includes a product of and/or resulting from machine learning that is used by the device to determine when and/or if to initiate a tinnitus management action. In an exemplary embodiment, this apparatus can be utilized to execute method action 39. In an exemplary embodiment, this device can be implemented in the above noted tinnitus management device 2177 and/or can be part of any of the prostheses detailed herein or any other device detailed herein providing that the art enables such. In an exemplary embodiment, this device can be a standalone device that provides output to a separate tinnitus masking device in signal communication therewith via the output of the device. In an exemplary embodiment, this device can be a standalone device that provides output to a hearing prosthesis, such as the hearing prostheses of FIG. 2, which output is received by the hearing prostheses and the hearing prosthesis is unable to receive the output and evaluate the output and activate a tinnitus mitigation/management regime, such as by way of example only and not by way of limitation, generating a tinnitus masking sound and/or altering a signal processing regime that eliminates certain frequencies and/or sounds or otherwise modifies such in a manner that is statistically significant vis-à-vis reducing and/or eliminating the likelihood of an occurrence of tinnitus.

In an exemplary embodiment, the aforementioned apparatus can be a palmtop computer that is in signal communication with a masking device or the like. That said, in an alternate embodiment, where the device is not a body carried portable device, the device can be a laptop computer or a desktop computer or the like. Still further, in an exemplary embodiment, the body carried portable device can be the hearing prosthesis of FIG. 2 and/or can be the tinnitus management device of FIG. 2C. In this regard, the phrase body carried portable device can be any device that is carried by the body, regardless of how such is carried. In an exemplary embodiment, the body carried device can be embodied in and/or a hearing prosthesis, a watch, or a wristband, or the like, and/or a pendant that hangs around the neck or the like.

Still, in an exemplary embodiment, the aforementioned apparatus can be a device that is structurally part of a tinnitus mitigation device and/or a hearing prosthesis as detailed herein and/or variations thereof. Indeed, the body carried portable device can be a hearing prosthesis or a tinnitus mitigation device.

The aforementioned input subsystem can be a subsystem that receives any one or more of the data associated with method 399 and variations thereof and/or other data detailed herein. In an exemplary embodiment, the input subsystem can be a wireless subsystem that received the data from another device and/or the input subsystem can be a wired subsystem that received the data from another device. In an exemplary embodiment, the input subsystem can be a wireless receiver and/or transceiver. The aforementioned output subsystem can be a transmitter and/or transceiver and/or can be a wired output subsystem that provides a signal to another device indicating whether or not to initiate a tinnitus management action with respect to the aforementioned product. By way of example only and not by way of limitation, the device can provide an output signal that initiates activation of the tinnitus management action. In this regard, the output from the output subsystem can be a control signal, and thus in an exemplary embodiment, the body carried portable device can be a control device or otherwise has control functionality. In an exemplary embodiment, this device can be part of the prosthesis of FIG. 2 or part of the tinnitus management device. Indeed, in an exemplary embodiment, the output subsystem can be the actual output of the device, which can be a masking sound or the like. In an alternate embodiment, output from the output subsystem can be a signal indicating that a tinnitus management action should be activated, but the signal does not control per se another device or activation of the device. The output can be data indicating that a tinnitus management action should be executed. In an embodiment of this exemplary embodiment, the receiving device can be a device that has logic that evaluates the signal and determines that it is a signal indicating that the tinnitus management action should be undertaken.

Exemplary embodiments include an apparatus, comprising a device (a body carried device or otherwise) including an input subsystem and an output subsystem, wherein the device includes a product of and/or resulting from machine learning that is used by the device to determine when and/or if to provide output using the output subsystem based on input into the input subsystem, wherein the device is at least part of a tinnitus management system. Exemplary embodiments include an apparatus comprising a body carried portable device including an input subsystem and an output subsystem, wherein the device includes a product of and/or resulting from machine learning that is used by the device to determine when and/or if to provide output using the output subsystem based on input into the input subsystem, wherein the device is at least part of a tinnitus management system.

In an exemplary embodiment, the product of an/or the arrangement resulting from machine learning is also used by the device to determine what type of tinnitus management action (e.g., from a plurality of actions) should be executed based on input into the input subsystem, wherein the management action at least one of remediates the effects of tinnitus or prevents a noticeable tinnitus scenario from occurring. By way of example only and not by way of limitation, the type of tinnitus management action can be a masking action or can be an adjustment to a hearing prosthesis setting that adjusts the sound processing in a manner that has been statistically significantly shown to reduce the likelihood of a tinnitus event occurring.

In an exemplary embodiment, preventing a recipient from noticing that he or she is experiencing a tinnitus episode can have utilitarian value in that in at least some instances, tinnitus is often worsened (or, more accurately, the perceived irritation associated therewith is often worsened) when the person realizes that the tinnitus is present.

Thus, in an exemplary embodiment, the device is configured to automatically initiate tinnitus masking using the product based on the input into the input subsystem.

It is briefly noted that while the embodiments detailed herein have been described in terms of a hearing prosthesis it is noted that the sound processing techniques thereof can also be utilized for other types of hearing devices, such as a headset or the like. By way of example only and not by way of limitation, tinnitus events can occur while a person is speaking on the telephone. In an exemplary embodiment, there can be a processor that processes the sound coming through the telephone in a manner that reduces the likelihood of a tinnitus effect occurring. Corollary to that is that a masking sound can be put through the telephone. The point is that any disclosure herein of a teaching associated with the hearing prostheses corresponds to an alternate embodiment of a non-hearing prosthesis (e.g., headset, telephone, stereo, other listening device, etc.) that utilizes that teaching as well.

Any tinnitus management action that can enable mitigation tinnitus and/or prevents a noticeable tinnitus scenario from occurring can be included in the actions detailed herein providing that the art enables such, and there is thus a device/system that is configured to do so.

In an exemplary embodiment, such as where the device is a structural part of a tinnitus mitigation device/the device is a tinnitus mitigation device, the output subsystem can be output that actually mitigates the tinnitus. Thus, in an exemplary embodiment, the product of and/or resulting from machine learning is used by the device to determine what type of output is to be outputted using the output subsystem based on input into the input subsystem, again wherein the output at least one of remediate the effects of tinnitus or prevents a noticeable tinnitus scenario from occurring. It is noted that mitigation includes reducing deleterious effects of tinnitus, including eliminating such, all relative to that which would otherwise be the case in the absence of the teachings herein/mitigation action. Such can be done by providing sound to the recipient/evoking a hearing percept in a different manner than that which would otherwise be the case, so as to emphasize or move frequencies so that the tinnitus does not interfere as much with the perception of the sound, thus making listening easier. Mitigation also includes masking. Mitigation can also include diverting a person's attention. The action of preventing a noticeable tinnitus scenario from occurring can be subjective or objective. In this regard, we refer to the above percentages applied for a six-month period. And note that those percentages can be applicable in some embodiments to the feature of the noticeable tinnitus scenarios.

In some embodiments, the input subsystem is configured to automatically obtain data indicative of at least physiological features past and/or present of a person who is using the device for tinnitus management purposes, and the input into the subsystem is the obtained data. By way of example only and not by way of limitation, the physiological features can go back less, than, equal to or greater than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 85, 90, 120, 150, 180 seconds 3.5, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 85, 90, 120, 150, or 180 minutes or more, or any value or range of values therebetween in 1 second increments (e.g., 4 minutes and 10 seconds, 123 minutes, 33 to 77 minutes, etc.). Any time frame that can enable the teachings detailed herein vis-à-vis the predictive features that can have utilitarian value can be utilized in at least some exemplary embodiments. In an exemplary embodiment, the input subsystem is configured to automatically obtain data indicative of at least ambient environmental conditions past and/or present of a person who is using the device for tinnitus management purposes and the input into the subsystem is the obtained data. The temporal features associated therewith can be those just detailed vis-à-vis the physiological features. Also, in an exemplary embodiment, the input subsystem is configured to automatically obtain data indicative of speech in an ambient environment past and/or present (again with any of the temporal features just detailed) and the device is configured to analyze the input and determined that the speech is likely speech that a user of the device seeks to understand, and the device automatically adjusts a tinnitus therapy based on the analysis.

It is noted that the aforementioned physiological features and/or the ambient environmental conditions can be those detailed above with respect to method 399 in some exemplary embodiments.

In an exemplary embodiment, the device is configured to log data indicative of at least one of ambient environmental conditions past and/or present of a person who is using the device for tinnitus management purposes or ambient environmental conditions past and/or present of a person who is using the device for tinnitus management purposes and the device is configured to correlate the logged data to tinnitus related events. In an exemplary embodiment, the data logging is used to train the expert system/establish the product. Thus, in an exemplary embodiment, the device “self-trains.” Additional details of the logging features and the self-training features will be described below, in conjunction with the training embodiments and the like of the expert system/trained network. For the moment, it is noted that embodiments of the results of the machine learning device that is utilized to predict tinnitus event and/or determine that a tinnitus event is occurring can be utilized in conjunction with the components that train the system in the first instance. Indeed, in an exemplary embodiment, the device can be a device that continuously or semi-continuously trains itself.

In at least some exemplary embodiments, the data logging and/or monitoring, at least the tinnitus episode related events (e.g., when the person is experiencing tinnitus and/or the characteristics thereof) can be executed utilizing manual methods of input and then after such, automated methods can then be implemented to manage the tinnitus or otherwise implement the tinnitus mitigation features detailed herein. Still, automatic methods of logging the data can be utilized. Indeed, in at least some exemplary embodiments, there can be no manual interaction with the devices that are utilized to log the data and/or to implement the tinnitus mitigation functions detailed herein, other than activating or deactivating the overall routine (and in some embodiments, the activation and deactivation can be automatic as well—such can be an embedded function in a hearing prosthesis for example that operates all the time unless the recipient of the prosthesis deactivates the function). Any device, system, and/or method that can enable a tinnitus pattern to be identified can be utilized in at least some exemplary embodiments.

Tinnitus patterns can correspond to the pattern of one set and/or the manifestation of the tinnitus (pitch, sharpness/dullness, etc.). Embodiments can focus on how loud a person perceives the tinnitus. All of this can be data that is provided into the systems herein that can be analyzed in at least some embodiments. The teachings detailed herein can be corrective or otherwise remedial to address a given manifestation in at least some exemplary embodiments.

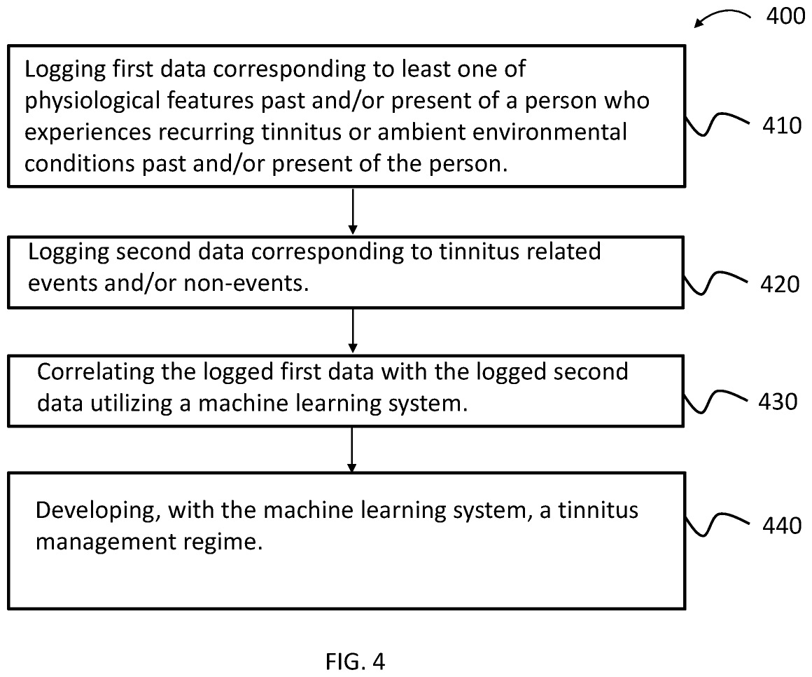

With respect to logging embodiments, FIG. 4 presents an exemplary flowchart for an exemplary method, method 400 according to an exemplary embodiment. As will be detailed below, the purposes of logging can be to obtain data sets that can be utilized by machine learning system as will be detailed below. With respect to the embodiment of FIG. 4, the data that is logged is correlated with other data utilizing a machine learning system. More specifically, method 400 includes method action 410, which includes logging first data corresponding to least one of physiological features past and/or present of a person who experiences recurring tinnitus or ambient environmental conditions past and/or present of the person. In an exemplary embodiment, method 400 is executed by a machine, while in other embodiments, this can be executed in a human based/manual manner. That is, it is noted that while at least some exemplary embodiments utilize machines or devices to log and/or classify the environment and/or physiological aspects, other embodiments utilize self-reporting and/or manual logging of such. Accordingly, any disclosure herein of data that is obtained or otherwise logged or otherwise captured by a machine also corresponds to a disclosure of data that is in an alternative embodiment tallied or otherwise logged manually. Indeed, in an exemplary embodiment, device 2140 can be utilized for self-reporting and the like. Still, some embodiments are directed towards a machine-based system/automated system.

In an exemplary embodiment, the data logging relates to ambient sound including speech of others and/or speech of the person who experiences the tinnitus episodes. In an exemplary embodiment, the data logging relates to any psychoacoustic data that can have utilitarian value with respect to enabling the teachings detailed herein. In an exemplary embodiment, the prosthesis that is being utilized to implement the teachings and/or another separate device, such as a device that is configured to capture sound, and record the sounds and/or evaluate the sounds and records the evaluation, can be utilized to achieve the data logging in whole or in part. As noted above, in at least some embodiments, scene classification can be utilized, and thus the data logging can include the utilization of scene classification techniques as detailed herein.

Moreover, it is noted that in at least some exemplary embodiments, the data logging entails monitoring the use of active tinnitus reduction methods and/or functions and determining when they are used by the person and/or how they are used, and correlating these against one or more ambient environmental conditions (which can include time of day) and/or physiological conditions and/or prosthesis settings or other device settings, etc., or any other factor that can influence tinnitus perception, or more accurately, any other factor that is statistically meaningful to influence tinnitus perception. In at least some exemplary embodiments, as detailed herein, the data that is logged is utilized by a machine learning system to learn and automatically apply a utilitarian tinnitus management or mitigation method, which can include reducing tinnitus (e.g., the tinnitus still present, but it is not as “severe” as otherwise might be).

Note also that while embodiments herein are disclosed as capturing sound and/or voice with a microphone or other sound capture device, and utilizing such for the data logging, it is noted that in alternative embodiments, voice and/or sound need not necessarily be captured. In this regard, in an exemplary embodiment, data relating to voice and/or sound is logged in a manual manner. Accordingly, any disclosure herein of capturing and/or data logging of voice and/or sound utilizing machine corresponds to the disclosure of an alternate embodiment where data associated with the voice and/or sound is self-reported or otherwise manually logged.

Thus, in at least some embodiments, the first data includes data indicative of speech of a person having tinnitus and/or speech of a person speaking to the person having tinnitus.

Data logging can be automatically executed in some embodiments. Some additional manners of implementation of such are described below. The point here is that any data that can enable the creation of a data set that can be utilized by machine learning system to implement the teachings detailed herein can be utilized in at least some exemplary embodiments.

Some additional examples of data logging or otherwise accumulating data to establish a data set that is utilized in the machine learning system will be described below. For the purposes of this immediate discussion, method action 410 is a method action that encompasses any data logging that can enable the teachings herein, utilizing any known technique that is available and that will provide utilitarian results.

Method 400 further includes method action 420, which includes logging second data corresponding to tinnitus related events and/or non-events. In this method action, the person afflicted with tinnitus can provide the data/can log the data himself or herself, or otherwise provide indications that he or she is or is not experiencing a tinnitus event. In this regard, in at least most circumstances, it will be the person who is afflicted with tinnitus who can tell whether or not he or she is having a tinnitus episode. Granted, there are some technologies that can detect that neurons are firing when they otherwise should not be/firing in an abnormal manner, and thus extrapolate that a tinnitus event is occurring. Typically, however, this requires an invasive device, such as an electrode array or a series of electrodes within the cochlea approximate thereto. Accordingly, while some embodiments do include utilizing non-affirmative input from the person afflicted with tinnitus to execute method action 420, most embodiments will typically rely upon self-reporting/self data logging by the person afflicted with tinnitus.

In some embodiments, this can be a simple regime of providing input into a system whenever the person affected with tinnitus has a tinnitus event and correlating such with time and/or with the first data that is logged. With respect to correlating such with time, if the logged first data is also correlated with time, which in some embodiments it is, the correlation between the two data can be executed by comparing like times or close enough like times or similar like times or any other regime that can enable the teachings detailed herein. In an exemplary embodiment, the recipient provides additional data beyond just the fact that he or she is experiencing a tinnitus episode. By way of example only and not by way of limitation, the person can provide input as to the severity and/or the perceived loudness and/or the frequency and/or the otherwise perception of the tinnitus. A predetermined scale can be utilized to describe the tinnitus. For example, a scale from 1 to 5 or scale from 1 to 10 can be utilized. With respect to determining a frequency, the devices, systems, and methods disclosed herein can have the feature that provides a series of tones at different frequencies where the person afflicted with tinnitus identifies the tone/frequency that is closest to the tinnitus perception. In an exemplary embodiment, the prosthesis and/or the tinnitus mitigation device or whatever device is being utilized can output different sounds of a predetermined frequency and the device can receive input, such as via an input button or the like from the recipient identifying the closest frequency. In an exemplary embodiment, the device can output a quasi-infinite number of frequencies and the recipient can iterate or otherwise match the closest frequency. A Newton Rapson method might be utilized to identify the frequency of the closest frequencies. A bracketing regime might be utilized. Any device, system, and/or method that can enable the characterization of the tinnitus perceived by the person afflicted with such can be utilized in at least some exemplary embodiments, and can be utilized as input with regard to method action 420.

In at least some embodiments, the devices, systems, and/or methods can characterize tinnitus based on the pitch and/or dullness and/or sharpness and/or the range of the tinnitus, the complexity and/or simplicity of the tinnitus, the temporal features thereof (e.g. momentary versus lengthy), the onset characteristics (sudden onset with loudness, slow onset gradually increasing with severity, etc.). In at least some embodiments, the data that is obtained can include data corresponding to any of these characteristics, generally received by input by the person of interest, and this data is then utilized in the analysis to develop the predictive algorithms, etc. Embodiments can automatically determine the characteristics of the tinnitus based on latent variables and initiate or otherwise apply a tinnitus mitigation regime based on those characteristics vs. other mitigation regimes that might be utilized for other characteristics.

To be clear, embodiments include devices, systems, and methods that enable a tinnitus mitigation regime to be tailored to a given individual's need, and this tailoring can be performed automatically. Note also that the tailoring can be directed towards what is desired to be mitigated versus other things that may not necessarily be desired to be mitigated. For example, certain frequencies may not be a problem for a person while other frequencies may be a problem at least when a cost-benefit analysis is performed with respect to the fact that certain mitigation regimes may have certain costs associated therewith.

In an embodiment, the person who is experiencing a real-time tinnitus episode can utilize one of the devices herein and activate the device to output sounds, where this device automatically outputs tones of increasing and/or decreasing frequency, and the recipient identifies the one or more frequencies that are perceived to be closest to the frequency. In an embodiment, the person afflicted with tinnitus can toggle between the frequencies to triangulate the frequencies of interest. This can be utilized in some of the data logging embodiments.

More specifically, in an exemplary embodiment, there can be a handheld or body carried device or a prosthesis or a tinnitus management device or any device that can enable at least some of the teachings detailed herein, including a smart phone or the like with an application there on, which device is configured to generate a short burst of audio at various pitch levels with different frequencies. In an exemplary embodiment, this can be pitch levels with different frequencies that are predetermined or otherwise have been identified as potentially at least having utilitarian value with respect to bracketing or otherwise focusing or identifying a given feature of the given recipient's tinnitus. These devices and/or systems can utilize a test module to play a short burst of the audio (it can be a variety of sounds including buzzing, ringing, chirping, hissing, whistling, etc.) to the user/person of interest, in response to which the user/person of interest indicates the frequency/frequencies that is closest to the tinnitus sound they are experiencing in the ear, by any of the various input regimes detailed herein (touch screen, speaking, etc.) at least some exemplary embodiments of these devices and/or systems are enabled to generate different pitches, modulations, and loudness to be able to mimic most (statistically speaking, and most includes all) tinnitus sensations. This allows the system to form a model of the tinnitus sensations, and so as to identify the best or otherwise a utilitarian means to address such. In an exemplary embodiment, this can correspond to data, such as physiological data, that is utilized in accordance with the teachings detailed herein, in an exemplary embodiment, can be utilized by the devices, systems, and/or methods detailed herein to identify or otherwise develop a tinnitus management regime has utilitarian value to the specific person of interest. By way of example only and not by way of limitation, the data that is obtained regarding the features of the person's tinnitus can be utilized in an automated system to identify outputs by a management system that can mask or otherwise mitigate or otherwise prevent the onset of tinnitus in the first instance. Note also that in an exemplary embodiment, this physiological data can be utilized in conjunction with other data (in a big data mode, for example) to identify certain scenarios that are statistically speaking more likely to create a tinnitus situation relative to others/more likely to trigger a tinnitus situation relative to others.

In an exemplary embodiment, the model is a map of tinnitus frustration levels and/or a map to appropriate countermeasures therefore, correlated to the various data inputs herein, so as to develop a tinnitus mitigation regime that has utilitarian value to an individual person who suffers from tinnitus.

Thus, in at least some exemplary embodiments, such embodiments enable the establishment of an automatic tinnitus modeler.

It is noted that method action 420 includes logging second data corresponding to nonevents as well. In this regard, there can be utilitarian value with respect to determining when the recipient is not experiencing a tinnitus event. Indeed, in an exemplary embodiment, the bulk of method action 420 entails logging non-tinnitus events. In an exemplary embodiment, the absence of input relating to a tinnitus event is at least sometimes declared a non-tinnitus event. Still, in some embodiments, the person afflicted with tinnitus can affirmatively provide input into a system or otherwise log that he or she is not experiencing a tinnitus event. Corollary to this is that a machine or other device that can sense the firing of neurons can be utilized to determine whether or not a tinnitus event is occurring, such as by determining that the neurons that are firing are indicative of neurons that should be firing with respect to the ambient noise environment.

Method 400 further includes method action 430, which includes correlating the logged first data with the logged second data utilizing a machine learning system. Some details of the use of machine learning are presented below. Briefly, in at least some exemplary embodiments, method action 430 is executed without any human interaction vis-à-vis the action of correlating. There could be human interaction with respect to providing the data to the machine learning system, but it is the machine learning system that performs the correlation of the data.

In an exemplary embodiment, this can be executed—indeed the entire method 400 can be executed—by any one or more of the devices detailed herein, including for example, the prosthesis of FIG. 2 or the tinnitus mitigation device of FIG. 2C, etc. Any device, system, and/or method that can enable the teachings detailed herein can be utilized in at least some exemplary embodiments and thus any device that can execute method action 430 or any the other method actions detailed herein, including the entirety of method 400 can be utilized in some embodiments.

As noted above, the second data can be tinnitus related events and/or non-events. The idea is that statistically significant factors may be present in the first data that can be correlated with the second data to determine that there is an increased likelihood of a tinnitus event occurring based on the existence of the first data. Utilizing the machine learning system can aid in identifying the statistically significant correlations. For example, if certain frequencies are prevalent at certain amplitudes shortly after the recipient has eaten lunch and the machine learning system determines that there is a statistically significant correlation between this and the occurrence of tinnitus at perceived frequency X, the occurrence of such fact pattern in the future may trigger a tinnitus mitigation action or some other action. It will be data that is utilized to prevent her in an attempt to prevent an onset of tinnitus or otherwise mask a tinnitus episode.

With respect to the nonevents, this can have utilitarian value with respect to identifying scenarios where tinnitus does not occur or is unlikely to occur. In this instance, if certain scenarios are present, and the scenarios are shown to be statistically unlikely to result in a tinnitus event, no action would be taken in at least some instances. That said, in an exemplary embodiment, it could be that the action taken is to try to keep the person afflicted in tinnitus in an environment where these scenarios exist. By way of example, if a background radio having sports talk is an environment where tinnitus is unlikely to occur, the management regime could include having sports talk radio in the background.

Any data in any correlation that can have utilitarian value with respect to identifying that there will be an onset of a tinnitus event and/or preventing or otherwise reducing the likelihood of the onset of tinnitus event can be utilized in at least some exemplary embodiments providing that the art enables such.

Method 400 further includes method action 440, which includes developing, with the machine learning system, a tinnitus management regime. Again, in an exemplary embodiment, this can be executed by any of the devices herein, and the result thereof can be utilized in such device. In this regard, at least some of the embodiments herein include self-taught devices that develop algorithms based on the first and second data and develop the tinnitus management regime utilized by the device. By way of example, the tinnitus management regime can be utilized to execute one or more of the actions of method 399 and/or can be utilized in the device described above that includes the product of the machine learning. Indeed, the product of machine learning can embody the tinnitus management regime.

Accordingly, the tinnitus management regime can be part of a trained system in at least some embodiments, and trained system is part of a portable device used to manage tinnitus.

That said, in some embodiments, the machine learning system is separate from the devices that are utilized to actually implement the tinnitus management regime. By way of example only and not by way of limitation, method action 440 can be executed with a standalone device that is not the possession and/or control of the person afflicted with tinnitus, but instead is under the control of a clinician or under the control of an organization completely separate from the person suffering from tinnitus. The tinnitus management regime developed by the machine learning system is then applied, whether device form or in a treatment method, separately.

Thus, in some embodiments, one or more of the actions of method 400 and/or all of method 400 is executed without involvement by a healthcare professional.

Some additional details of implementing machine learning and devices associated therewith, including data logging, will be describe below. First, however, some additional features of method 400 will be presented.

In an exemplary embodiment, the tinnitus management regime that results from method action 440 includes one or more sounds that mask the tinnitus, which one or more sounds are identified via the action of developing that method action 440. In an exemplary embodiment, the tinnitus management regime can include one or more stimulations that are applied to a recipient that mitigate tinnitus. In an exemplary embodiment, the results of the correlation of method action 430 can identify the frequencies of tinnitus that statistically significantly occur in a scenario that corresponds to a scenario extrapolated from the first data. Thus, the one or more sound that masks the tinnitus can be sounds having frequencies that will mask the identified frequencies of the tinnitus, or at least are likely to mask the frequencies of the tinnitus, as compared to other frequencies of the masking sounds. That said, in some embodiments, the tinnitus management regime is more based on the temporal application of the masking sounds and/or the initiation of the masking sounds in the first instance based on an extrapolated scenario that is statistically linked to the onset of a tinnitus event.

To be clear, while some embodiments are focused on masking sounds, other embodiments can include additional types of mediation and/or may not necessarily utilize masking sounds. Any tinnitus management actions that can be utilized in the tinnitus management regime that can have utilitarian value for mitigating or otherwise managing tinnitus can be utilized in at least some exemplary embodiments providing that the art enables such.

In an exemplary embodiment, any of the devices herein, such as the smart phone can be configured accordingly, and can evaluate data input and automatically trigger the playing of background sounds/music/noise through its speakers, or stream the sounds to wireless earbuds (or mix in the background sounds to the currently streamed audio) to mitigate the tinnitus.

Thus, in an exemplary embodiment, the tinnitus management regime includes triggering one or more actions and/or advisories, where a basis for the action of triggering is identified via the action of developing that method action 440. An example of the advisory may be to have the recipient leave a room in which he or she is located or otherwise change of venue and/or eliminate a sound resource of sound or otherwise reduce the amount of sound that is being received by the recipient (e.g., using ear plugs or ear mufflers) and/or having the person at issue undertaking some form of exercise or some form of movement, etc. Any action and/or advisory that can have utilitarian value with respect to managing tinnitus can be utilized in at least some exemplary embodiments providing that the art enables such.

As detailed above, in some embodiments, the teachings detailed herein are implemented with respect to a person that has a hearing prosthesis, such as for example, the device of FIG. 2 or any the other devices disclosed herein. Accordingly, in an exemplary embodiment, the first data includes data indicative of a hearing prosthesis device setting. In an exemplary embodiment, the machine learning system identifies a correlation between device settings and the onset of tinnitus and/or the lack of onset of tinnitus. Accordingly, there can be utilitarian value with respect to the first data being hearing prosthesis device settings. The settings could be volume gain noise cancellation, beamforming, or any other setting that has a statistical correlation between tinnitus and/or lack of tinnitus.

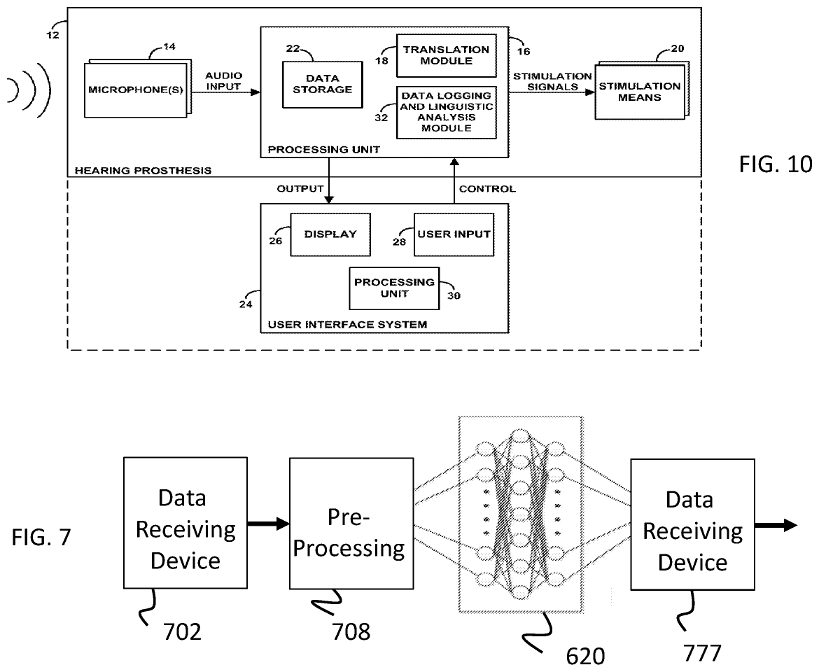

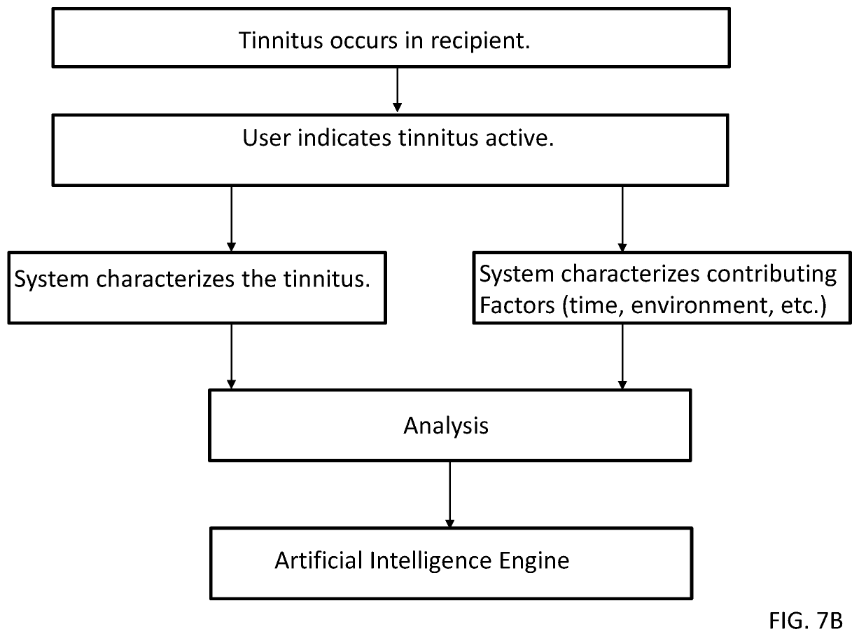

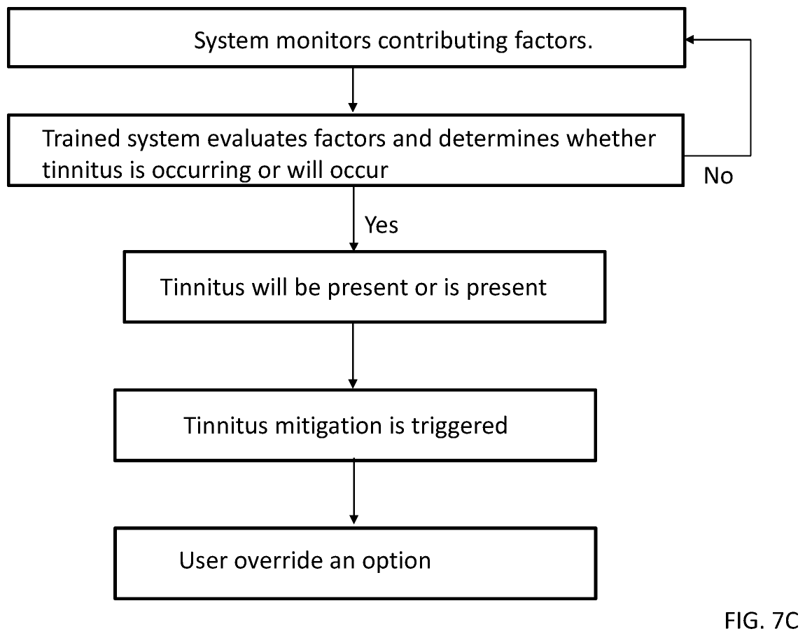

FIG. 7B presents a brief exemplary flowchart for an exemplary learning phase of an artificial intelligence device or otherwise neural network device according at least some embodiments. FIG. 7C presents a brief exemplary flowchart for the implementation phase of the trained artificial intelligence device or otherwise neural network device according to at least some embodiments.

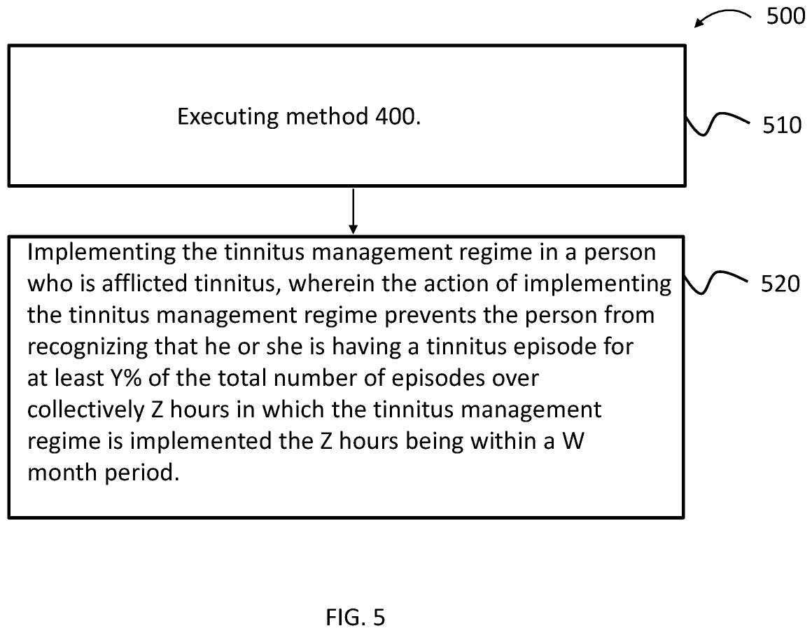

FIG. 5 provides another exemplary flowchart for an exemplary method. In an exemplary embodiment, there is a method, method 500, that includes method action 510, which includes executing method 400. Method 500 further includes method action 520, which includes the action of implementing the tinnitus management regime in a person who is afflicted tinnitus, wherein the action of implementing the tinnitus management regime prevents the person from recognizing that he or she is having a tinnitus episode for at least Y % of the total number of episodes over collectively Z hours in which the tinnitus management regime is implemented the Z hours being within a W month period. In an exemplary embodiment Y is at least and/or equal to 20, 25, 30, 35, 40, 45, 50, 55, 60, 65, 70, 75, 80, 85, 90, 95, or 100%, or any value or range of values therebetween in 1% increments.

Embodiments also include an exemplary system as follows. The system can include a sound capture apparatus (e.g., microphone) configured to capture ambient sound, concomitant with the embodiments detailed above. In an exemplary embodiment, the sound capture apparatus can be utilized in conjunction with the data logging actions to capture ambient sound. An exemplary embodiment, the devices and systems herein are configured to record sound (constantly and/or when as needed or utilitarian or a weighted basis) which recording can be utilized for ultimate data logging. Such can be done in accordance with PCT Application Publication No. WO 2020/021487, published on Jan. 30, 2020, entitled Habilitation and/or Rehabilitation Methods And Systems. That said, in an exemplary embodiment, the sound capture apparatus is simply a sound capture apparatus utilized for hearing prostheses in a traditional manner. The system further includes an electronics package (computer chip, processor, or any of those detailed herein and variations thereof) configured to receive data based on at least an outputted signal from the sound capture apparatus and analyze the data to determine based thereon that there exists a statistical likelihood of a future tinnitus event in the near term of a person using the system. Again, in an exemplary embodiment, the electronics package is a results of machine learning. In another exemplary embodiment, the electronics package is a conventional circuit (microprocessor or otherwise) established by firmware and/or that utilizes software that analyzes the data from the microphone and determines the aforementioned statistical likelihood. In an exemplary embodiment, the sound capture apparatus is part of a separate device from a device that includes the electronics package. In an exemplary embodiment, the electronics package can be the smart phone 2140. In an exemplary embodiment, the electronics package can be a device that is remote from the sound capture apparatus in a big way, such as being located far away such that the Internet and/or a cell phone or a telephone or some other communication system is needed to communicate with such (from the location of the sound capture apparatus). Conversely, in some embodiments, the sound capture apparatus and the electronics package are part of a single same physical device, which can correspond to a prosthesis corresponding to the device of FIG. 2 and/or the tinnitus mitigation device of FIG. 2C.

In an exemplary embodiment, the system is configured to automatically initiate an output that preemptively reduces the likelihood of the future tinnitus event upon the determination. In an exemplary embodiment, the output can be a masking sound, where the output could be a recommendation to the person of interest to do something, such as eliminate a background noise or perform some exercise (perhaps breathing exercise) or to make some change or activate something that reduces the likelihood of future tinnitus event. In an exemplary embodiment, this can be audible instructions/recommendations utilizing the output speaker of the prosthesis, this could be a visual instruction utilizing the display screens of the smart phone or the display screen of the tinnitus mitigation device 2177, or any other way of communicating such to the recipient. It is noted that the automatic initiation of an output can be an action that corresponds to the electronics package being remote from the person of interest, an electronics package providing output that is communicated over the Internet or the like to the person of interest, or more accurately, to a device in the possession of the person of interest/person using the system.

In an exemplary embodiment, the system is configured to automatically initiate the output without affirmative input from the person of interest/person using the system. This is concomitant with the embodiments detailed above. That said, in some embodiments, the system is configured to initiate the output in conjunction with affirmative input from the person of interest. In an exemplary embodiment, this can be input indicating that the person is experiencing tinnitus and/or the type of tinnitus and/or the severity of tinnitus. In an exemplary embodiment, this can be input indicating that the person, for whatever reason, believes that a tinnitus episode is imminent or likely to occur (intuition for example).

Indeed, in an exemplary embodiment, the input can be input distinguishing between one of the other. In this regard, embodiments of the teachings detailed herein can take different actions with respect to whether or not a tinnitus episode is occurring versus whether or not a tinnitus episode is predicted to occur. By way of example only and not by way of limitation, in an exemplary embodiment, if the tinnitus episode is occurring (or, more accurately, a determination is made such is occurring) a masking function may be initiated. Conversely, only by way of example and not by way of limitation, in an exemplary embodiment, if the tinnitus episode is predicted to occur, but has not yet occurred, a setting might be changed on a hearing prosthesis (automatically or a recommendation might be given to the person) or certain noise cancellation routines might be implemented/engaged, which noise cancellation has been shown in a statistically significant manner to reduce the likelihood of the occurrence of tinnitus, etc.

In an exemplary embodiment of the systems detailed herein, the data received by the electronics package further includes data based on physiological data relating to the person, and the electronics package is configured to evaluate the data based on physiological data in combination with the data based on the outputted signal an determine based thereon that there exists a statistical likelihood of a future tinnitus event in the near term of a person using the system. Thus, in this exemplary embodiment, the data that is evaluated can be data based on sound scene classification as well as physiological data. That said, such is not limited to sound scene classification, but other types of processing associated with captured sound to be utilized in at least some exemplary embodiments.

In some exemplary embodiments, the electronics package includes logic that applies a dynamic and individualized probability metric to determine that there exists the statistical likelihood of a future tinnitus event in the near term of a person using the system. In an exemplary embodiment, concomitant with the logging embodiments detailed above, the system is configured to automatically log data indicative of at least one of ambient environmental conditions past and/or present of the person or physiological conditions past and/or present of the person, and the system is configured to automatically correlate the logged data to tinnitus related events of the person and automatically develop a tinnitus management regime. This can be done by machine learning as detailed herein. Moreover, the electronics package is configured to execute the tinnitus management regime to analyze the data to determined based on the data that there exists the statistical likelihood of the future tinnitus event in the near term of the person using the system.

Accordingly, in an exemplary embodiment, there are devices, systems, and/or methods that are configured to activate and apply tinnitus masking automatically through the dynamic and individualized probability metric system.

An exemplary embodiment can include a system that comprises a tinnitus onset predictive subsystem (such as for example the product that results from machine learning, or a program processor/processor that has access to software that enables production of tinnitus onset, etc.) and a tinnitus management output subsystem. In an exemplary embodiment, the system further comprises a tinnitus onset predictive metric development subsystem. Consistent with the detailed of at least some exemplary embodiments presented herein, in some exemplary embodiments, system includes a trained neural network, wherein the trained neural network is part of the tinnitus onset predictive subsystem and the tinnitus onset predictive metric development subsystem contributes to the training of the trained neural network. Further, in at least some exemplary embodiments, the tinnitus onset predictive subsystem is an expert sub-system of the system that includes a code of and/or from a machine learning algorithm to analyze data relating to a user of the system in real time and wherein the machine learning algorithm is a trained system trained based on a statistically significant population of tinnitus afflicted persons. In at least some embodiments, the tinnitus onset predictive subsystem is configured to automatically analyze a linguistic environment metric in combination with a non-linguistic environment metric correlated to the linguistic environment metric, all inputted into the system, and based on the analysis, automatically determine whether or not a tinnitus event is imminent. Still further, in an exemplary embodiment, the system is configured to identify speech of a user of the system and the linguistic environment metric is the speech of the user.

At least some embodiments can also take the entire psychoacoustic characteristics of both ears of a person who suffers from tinnitus into consideration. In an exemplary embodiment, a person who suffers from tinnitus may happen to be a bilateral recipient or a bimodal hearing device user. The devices and/or systems and/or methods detailed herein can be configured or otherwise are implemented to consider a scenario that while applying a certain masking or other tinnitus mitigation stimulus at certain frequencies to one ear, in order to maintain an optimal hearing perception for the individual, the system can consider enhancing amplitude and/or changing a dynamic range of certain settings of those frequencies for the other ear.

Indeed, the features of the paragraph immediately above need not necessarily be restricted to only hearing aid users/to people who have hearing problems (aside from tinnitus to the extent such is considered a hearing problem). By way of example only and not by way of limitation, the device of FIG. 2 detailed above can be located one day left ear, and another device also corresponding to FIG. 2 detailed above can be located on the right ear, and to the extent that a masking or the like or some other sound is applied to one ear, the prostheses for that ear could implement such, and the other prostheses could implement sound processing that could counterbalance the stimulus applied to the “treated” ear. That said, it is noted that in some embodiments, earplugs or earphones the like can be utilized instead of full prostheses such as that in FIG. 2. The point is, in at least some exemplary embodiments, bilateral and/or bimodal implementation regimes can be utilized in some embodiments, where one ear can be utilized as a counterbalance to tinnitus mitigation stimulation that is applied to another ear.

Corollary to this is that in at least some exemplary embodiments, the devices, systems, and methods enable the identification of which ear a tinnitus event is occurring or otherwise is likely to occur based on the data that is obtained. Indeed, in some embodiments, a determination can be made that there is a statistical likelihood that tinnitus event will occur in one ear versus another ear based on the data that the system obtains/utilizes.

As noted above, embodiments include evaluating an auditory environment and/or data logging and auditory environment. In an exemplary embodiment, this can correspond to measuring an auditory environment (auditory scene analysis and data logging). Auditory scene analysis can involve a classification and decision-making process that can recognize a wide variety of auditory environments, and systems detailed herein can be configured to evaluate such and initiate a tinnitus mitigation action and/or identify a species of tinnitus mitigation action that has more utilitarian value with respect to another action, and initiate such. Through data logging, the systems can collect and store data over a period of time in order to enable the analysis of specific trends or record data-based events/actions in the individual's real world auditory environment. This can, in some embodiments, inform evaluation of scenarios that can result in tinnitus events, and based on such, can enable the systems that predict/determine the occurrence of such and/or the characterization of such.

As noted above, embodiments can rely on own voice detection in that the tinnitus mitigation actions may be triggered based on an analysis of a person's own voice (the person suffering from tinnitus). In an exemplary embodiment, own voice detection is executed according to any one or more of the teachings of U.S. Patent Application Publication No. 2016/0080878 published on Mar. 17, 2016, entitled Control Techniques Based on Own Voice Related Phenomena, and/or the implementation of the teachings associated with the detection of the invoice herein are executed in a manner that triggers the control techniques of that application. Accordingly, in at least some exemplary embodiments, the devices and systems can be configured to or otherwise include structure to execute one or more or all of the actions detailed in that patent application. Moreover, embodiments include executing methods that correspond to the execution of one or more of the method actions detailed in that patent application.

In an exemplary embodiment, own voice detection/detection of the user (and by extension, differentiation of other voices—if it is not the user's voice, it must be that of another) is executed according to any one or more of the teachings of WO 2015/132692 entitled Own Voice body Conducted Noise Management, published on Sep. 11, 2015, and/or the implementation of the teachings associated with the detection of the user (own) voice herein are executed in a manner that triggers the control techniques of that application. Accordingly, in at least some exemplary embodiments, the various devices and/or systems detailed herein are configured to or otherwise include structure to execute one or more or all of the actions detailed in that patent application. Moreover, embodiments include executing methods that correspond to the execution of one or more of the method actions detailed in that patent application.

It is noted that in at least some exemplary embodiments, there is a correlation between the data logging and the voice that is captured. That said, in some alternate embodiments, there is no correlation between the data logging in the voice that is captured. In this regard, in an exemplary embodiment, the teachings detailed herein that utilize the captured voice or the data associated with the captured voice as well as the logged data can utilize such even though there is no correlation between the two.

An alternate embodiment includes a method, comprising capturing an individual's voice with a machine and logging data corresponding to events and/or actions of the individual's real world auditory environment, wherein the individual is speaking while using a hearing assistance device, and the hearing assistance device at least one of corresponds to the machine or is a device used to execute the action of logging data.

By hearing assistance device, it is meant a hearing prosthesis as well as a device that simply will help someone here, such as a device that is utilized with a smart phone and a headset or the like, which is not a hearing prosthesis. Indeed, in some embodiments, the hearing assistance device could be an amplified telephone. Any teaching herein can be combined/implemented with a hearing assistance device according to some embodiments.

It is briefly noted that while the above recent paragraphs are directed towards and auditory environment, the teachings herein also include non-auditory environments as well, such as any of those detailed herein. Accordingly, any device, system, and/or method that can enable the data logging or recording of any utilitarian aspect of a person's environment can be utilized in at least some exemplary embodiments. By way of example only and not by way of limitation, cameras, heart rate monitors (Fit Bit™ type devices), temperature monitors, exercise monitors, movement monitors, blood pressure monitors, EKG monitors, EEG monitors, global positioning systems, etc., can all be utilized in some embodiments to obtain data indicative of what those monitors are used for, and devices can include recording the obtained data.

With respect to embodiments that utilize to logged data, in at least some exemplary embodiments, the logged data can be based on the captured sound that is captured by the machine or by another device, and thus can also be based on another source other than the machine. In an exemplary embodiment, a hearing assistance device or any other device herein can be utilized to capture and ambient sound environment, and such can be a hearing prosthesis, and such can be a machine that is utilized to capture the individual's voice and/or the voice of others and/or the ambient auditory environment. In an exemplary embodiment, the hearing assistance device is not a hearing prosthesis, but is still the machine that is utilized to capture the individual's voice. In an exemplary embodiment, irrespective of whether or not the hearing assistance device is a hearing prosthesis, another device other than the hearing assistance device is utilized to capture the individual's voice and/or the voice of others and/or the ambient sound environment.

Some exemplary embodiments rely on statistical models and/or statistical data in the variation evaluations detailed herein and/or variations thereof. The “nearest neighbor” approach will be described in greater detail below. However, for the moment, this feature will be described more broadly. In this regard, by way of example only and not by way of limitation, in an exemplary embodiment, the evaluation of data associated with the ambient environment and/or physiological features includes comparing such for the person of interest with similarly situated people. In an exemplary embodiment, the statistically significant group can include, for example, ten or more people who speak the same language as the recipient and who are within 10 years of the age of the recipient (providing that the recipient is older than, for example, 30 years old, in some instances by way of example only and not by way of limitation), the same sex as the recipient, etc.

In an exemplary embodiment, a machine learning system, such as a neural network, can be used to analyze the data of the statistically significant group so as to enable (or better enable) the comparison/correlation. That said, in some exemplary alternate embodiments, the comparison of the data associated with the person of interest can be performed against a statistically significant data pool of other tinnitus sufferers who are similarly situated.

While the embodiments detailed above have been described in terms of comparing the data of the person of interest to a statistically significant group/a model of a statistically significant population, in some other embodiments, the evaluation of the data can be executed without the utilization of statistical models.

Thus, as seen from the above, in an exemplary embodiment, embodiments can include any convenient or otherwise available or otherwise modifiable consumer electronics device and/or prosthesis device and/or tinnitus mitigation device that can include an expert sub-system that includes code of and/or from a machine learning algorithm to analyze metrics having utilitarian value with respect to implementing the teachings detailed herein that are based on input into the device (or system), and wherein the machine learning algorithm is a trained system. The device and/or system can be trained based on the individual experiences of the person that utilizes the device and/or system and/or can be trained based on a statistically significant population of tinnitus sufferers (more on this below).

An exemplary machine learning algorithm can be a DNN, according to an exemplary embodiment. In at least some exemplary embodiments, the input into the system can be processed by the DNN (or the code produced/from by the DNN).

Embodiments thus include analyzing the obtained data/input into the system utilizing a code of and/or from a machine learning algorithm to develop data that can be utilized to implement the applicable teachings herein. Again, in an exemplary embodiment, the machine learning algorithm can be a DNN, and the code can correspond to a trained DNN and/or can be a code from the DNN (more on this below). It is noted that in some embodiments, there is no “raw data”/“raw ambient environment data” input into the devices and/or systems in general, and the DNN in particular. Instead, some or all of this is pre-processed data. Any data that can enable the system and/or device and/or the DNN or other machine learning algorithm to operate can be utilized in at least some exemplary embodiments.

It is noted that any method action disclosed herein corresponds to a disclosure of a non-transitory computer readable medium that has program there on a code for executing such method action providing that the art enables such. Still further, any method action disclosed herein where the art enables such corresponds to a disclosure of a code from a machine learning algorithm and/or a code of a machine learning algorithm for execution of such. Still as noted above, in an exemplary embodiment, the code need not necessarily be from a machine learning algorithm, and in some embodiments, the code is not from a machine learning algorithm or the like. That is, in some embodiments, the code results from traditional programming. Still, in this regard, the code can correspond to a trained neural network. That is, as will be detailed below, a neural network can be “fed” significant amounts (e.g., statistically significant amounts) of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm that can be utilized separately from the trainable neural network. In one embodiment, there is a path of training that constitutes a machine learning algorithm starting off untrained, and then the machine learning algorithm is trained and “graduates,” or matures into a usable code—code of trained machine learning algorithm. With respect to another path, the code from a trained machine learning algorithm is the “offspring” of the trained machine learning algorithm (or some variant thereof, or predecessor thereof), which could be considered a mutant offspring or a clone thereof. That is, with respect to this second path, in at least some exemplary embodiments, the features of the machine learning algorithm that enabled the machine learning algorithm to learn may not be utilized in the practice some of the method actions, and thus are not present the ultimate system. Instead, only the resulting product of the learning is used.

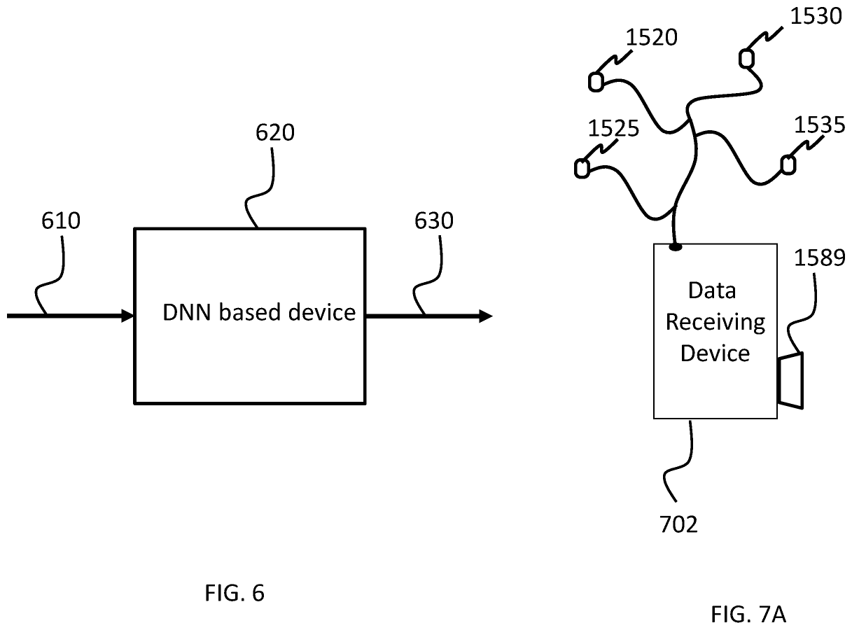

FIG. 6 depicts an exemplary conceptual functional black box schematic associated with method action 392 are any of the other method actions detailed herein by way of example, where input 610 is input into a DNN based device 620 that utilizes a trained DNN or some other trained learning algorithm or trained learning system (or the results thereof—in an exemplary embodiment, the product of machine learning—as used herein can correspond to a trained learning algorithm or trained learning system as used in operational mode after training has ceased and product of machine learning can correspond to a product that is developed as a result of training—again, this will be described in greater detail below), and the output is a signal 630 that is provided to a person suffering from tinnitus and/or to a tinnitus mitigation device, or a system that is configured for such, such as a hearing prosthesis designed accordingly, which signal activates tinnitus mitigation functions of that device. In this exemplary embodiment, device 620 can be a processor or a chip or any electronics or circuitry that can enable the teachings detailed herein, providing that such is configured to do so.

It is noted that in at least some exemplary embodiments, the input 610 comes directly from a microphone, while in other embodiments, this is not the case. In an exemplary embodiment, the input comes from any of the other monitoring devices detailed herein or any other monitoring device that can enable the teachings detailed herein. In some embodiments, the input 610 comes directly from these components/monitoring devices, and in an exemplary embodiment, there is a body device or a body carried device that includes any one or more of these monitoring devices or devices that are configured to enables such monitoring, etc. This body carried device can also be a device that has the tinnitus mitigation features detailed herein. That said, in an exemplary embodiment, this body carry device can be a device that is solely dedicated to obtaining the data for data logging purposes, where, in an exemplary embodiment, after the data logging occurs, there is no more data logging that is executed and/or the tinnitus mitigation devices are devices that are configured based on the data logged but the device does not need data logging. That said, in an exemplary embodiment, the body carry device can be a device that is utilized to obtain data indicative of an ambient environment and/or of the physiological features of the person at issue. In an exemplary embodiment, this can be a dedicated device that is in signal communication with a device that initiates the tinnitus mitigation and/or applies a stimulus to the recipient to mitigate tinnitus. This device that initiates the tinnitus mitigation and/or applies the stimulus can be a device that receives data from this body worn/body carry device and analyzes the data according to the teachings detailed herein.

Going back to the device 620, in an exemplary embodiment, this can be a device that is located remotely from the sensors and/or from where the data was collected, the data being communicated via a communication system such as the Internet or the like.

Input 610 can correspond to any input that can enable the teachings detailed herein to be practiced providing that the art enables such. Thus, in some embodiments, there is no “raw sound” input and/or no raw ambient environment input and/or no raw physiological data input into the DNN. Instead, some or all of this can be all pre-processed data. Any data that can enable the DNN or other machine learning algorithm or system to operate can be utilized in at least some exemplary embodiments.

It is noted that at least some embodiments can include methods, devices, and/or systems that utilize a DNN inside a prosthesis and/or inside a tinnitus mitigation device and/or along with such (including a smart phone or a computer, etc.). In some embodiments, a neural network, such as a DNN, is used to directly interface to the audio signal coming from one or more microphones and/or to directly interface to the data signal coming from one or more of the other monitoring devices detailed herein, process this data via its neural net, and determine whether or not the environmental conditions and/or the physiological conditions correspond to those which in the past have been indicative of a forthcoming tinnitus event of the person associated with the method and/or that these conditions correspond to a current tinnitus event and process. The network can be, in some embodiments, either a standard pre-trained network where weights have been previously determined (e.g., optimized) and loaded onto the network, or alternatively, the network can be initially a standard network, but is then trained to improve specific person results.

FIG. 7 presents an exemplary system for executing at least the method 399. As seen, there is a data receiving device 702, which can be a microphone and/or a Fit Bit™ device or a device that has similar functionality and/or the same functionality that is in real-time signal communication with one of the devices herein, or EKG or body temperature measuring devices or a GPS receiver or any of the monitoring devices disclosed herein or any others that can enable the teachings herein, that can execute method action 390. As seen, there can be a preprocessing component 708, which can be optional, and can include a digital to analog converter or an analog-to-digital converter or any other device that can preprocess the results from the data receiving device in a manner that is utilitarian for device 6202 receive. Thus, collectively, devices 702 and 708 execute method action 390.

FIG. 7A provides a brief conceptual version of data receiving device 702, that includes read electrode(s) 1520 and temperature sensor 1530, the former being able to, by way of example only and not by way of limitation, measure electrical impulses, in the body (EEG or EKG), the latter being able to measure body temperature. Also shown is a blood pressure sensor 1525 and a perspiration sensor 1535. Any other sensor that can enable the recordation of physiological features can be utilized in some embodiments. The various sensors provide an interface between the person at issue in the overall data receiving device. Also shown is microphone 1589. Microphone 1589 is configured to capture and/or monitor the ambient auditory environment, such as a background ambient audio environment. In an exemplary embodiment, there can be two or more microphones, and the overall arrangement can have a beamforming and/or sound origination location feature, which can provide data that is utilized with the devices methods and/or systems detailed herein.

In an exemplary embodiment, any one or more of the sensing/monitoring arrangements of PCT patent application publication number WO 2020/089856, published on May 7, 2020, and also any of the physiological features that are monitored or otherwise measured in that application can be utilized in at least some exemplary embodiments herein providing that such is utilitarian in the art enables such. Any one or more of the sensing/monitoring arrangements can be part of the input device 702.

The output from devices 702 and/or 708 corresponds to neural network inputs so as to be obtained by device 620. In at least some exemplary embodiments, the network will have already been loaded with pre-taught weights (more on this below). The neural network of device 620 (which can be a deep neural network that perform signal processing/audio processing/light processing, etc.) then determines whether or not a tinnitus episode is statistically likely to occur in the short run and/or whether or not a tinnitus episode is occurring and/or what type of stimulus should be provided to the person who suffers from tinnitus to prevent and/or mask the tinnitus episode. Results of this are provided to data receiving device 777, which can correspond to the tinnitus mitigation device and/or a processor or a sub processor of a hearing prosthesis or any other device that can controllably provide stimulation to a person suffering from tinnitus. In an exemplary embodiment, the data receiving device can be a processor or a computer chip or an electronic circuit that receives the input from the neural network device 620, and controls and output accordingly. In an exemplary embodiment, the data receiving device can be a device that is configured to provide audio and/or visual output to a person suffering from tinnitus, which output can be a recommendation or instruction to do something, such as eliminate a certain sound or move from a given area, so as to avoid the onset of tinnitus or otherwise reduce the severity of a current tinnitus episode, etc.

It is to be noted that in an exemplary embodiment, devices 620 and 777 can be combined in a single device. Corollary to this is that in an exemplary embodiment, device 620 can be remote from device 777. In an exemplary embodiment, device 620 can communicate with device 777 over the Internet or the like, and device 777 can be the prostheses detailed above. In an exemplary embodiment, device 620 can be embedded in/be part of the prostheses detailed herein or other devices detailed herein, such as the tinnitus mitigation device noted above.

More specifically, in an exemplary embodiment, device 620 is a microprocessor or otherwise a system that includes the product from the machine learning. In an exemplary embodiment, device 777 can include/be circuitry that may include logic circuits that receives the output from the processor 620 and applies the tinnitus mitigation actions accordingly. In this regard, mapping section 540 can correspond to a processor of a cochlear implant. Indeed, in an exemplary embodiment, a hearing prosthesis can be obtained, and device 620 can be inserted in between the sound capture arrangement thereof and the output thereof/a sound processor thereof. In an exemplary embodiment, there can be a processor of a hearing prosthesis or of any other device disclosed herein and the processor could be modified to include the features associated with device 620, or otherwise can include a separate processor that communicates with the processor of a hearing prosthesis/hearing prosthesis sound processor, to execute the actions associated with device 620. (It is noted that in an alternate embodiment, processor 620 is replaced with a non-processing device, or includes non-processing devices, such as a chip or the like that is a result of a machine learning algorithm or machine learning system, etc. Any disclosure herein of a processor corresponds to a disclosure in an embodiment of a non-processor device or a combined processor-non-processor device where the non-processor is a result of machine learning.)

In an exemplary embodiment, device 620 and device 777 are all part of a single processor. In an exemplary embodiment, device 708, 620 and 777 are all part of a single processor. Thus, in an exemplary embodiment, there is a processor that is programmed and configured or otherwise contains code or circuitry or switches, etc., to execute one or more of the functionalities detailed herein.

In an exemplary embodiment, the aforementioned processor is a general-purpose processor that is configured to execute one or more of the functionalities herein. Again, in some embodiments, the processor includes a chip that is based on machine learning/from machine learning. In an exemplary embodiment, the aforementioned processor is a modified cochlear implant sound processor that has been modified to execute one or more of the functionalities detailed herein, such as via the inclusion of an ASIC developed as a result of machine learning. In an exemplary embodiment, a solid-state circuit is configured to execute one or more of the functionalities detailed herein. Any device, system, and/or method that can enable the teachings detailed herein can be utilized in at least some exemplary embodiments.

It is noted that in an exemplary embodiment, the device 620 can reside or otherwise be on the smart device 2140 detailed above. In an exemplary embodiment, the processor of the smart device can have the functionality via programming or the like of device 620. In an exemplary embodiment, the microphone of the smart device corresponds to data receiving device 702, and the processing chain all the way to the output of 777 can be executed by the smart device 2140. Thus, in an exemplary embodiment, there is a smart device that is configured to execute one or more of the functionalities associated with these components. In an exemplary embodiment, the smart device can be the device that provides the stimulus to the person who suffers from tinnitus to mask and/or reduce the likelihood of an occurrence of the tinnitus onset or otherwise to provide instructions recommendations to that person, etc.

In at least some exemplary embodiments, the devices and/or systems herein can operate in different modes so that the tinnitus management functionalities are activated and/or deactivated. First, it is noted that in at least some exemplary embodiments, the activities of the DNN can be controlled or otherwise selectively enabled and/or disabled. By way of example only and not by way of limitation, in some embodiments, the devices disclosed herein and/or systems disclosed herein and variations thereof, such as the hearing prostheses detailed herein, can operate as a normal traditional device, such as a normal traditional hearing prosthesis even while using the DNN, and in other embodiments, the DNN and can be selectively enabled or disabled, where the disabled DNN results in the normal operation of the device, such as the normal sound processor operating in a normal manner. Conversely, the prosthesis can be controlled to enable the DNN to do its thing. Moreover, in some embodiments, the DNN can be selectively controlled to operate differently.

Some embodiments can utilize any form of the genus known as artificial intelligence to execute one or more of the functionalities and/or method actions detailed herein providing that the art enables such as otherwise noted. The teachings above are generally focused on neural networks. In at least some exemplary embodiments, a deep neural network, such as a back propagated deep neural network, is utilized. It is noted that in some other embodiments, other types of artificial intelligence are utilized, such as by way of example only and not by way of limitation, expert systems. That said, in some embodiments, the neural network is specifically not an expert system, consistent with the fact that any disclosure of any embodiment herein constitutes a corresponding disclosure of an embodiment that specifically does not have that embodiment.

Any learning model that is available and can enable the teachings detailed herein can be utilized in at least some exemplary embodiments. As noted above, an exemplary model that can be utilized with voice analysis and other audio tasks is the Deep Neural Network (DNN). Again, other types of learning models can be utilized, but the following teachings will be focused on a DNN.

There are many packages now available to perform the process of training the model. Simplistically, the input measures are provided to the model. Then the outcome is estimated. This is compared to the subject's actual outcome, and an error value is calculated. Then the reverse process is performed using the actual subject's outcome and their scaled estimation error to propagate backwards through the model and adjust the weights between neurons, and improving its accuracy (hopefully). Then a new subject's data is applied to the updated mode, providing a (hopefully) improved estimate. This is simplistic, as there are a number of parameters apart from the weight between neurons which can be changed, but generally shows the typical error estimation and weight changing methods for tuning models according to an exemplary embodiment.

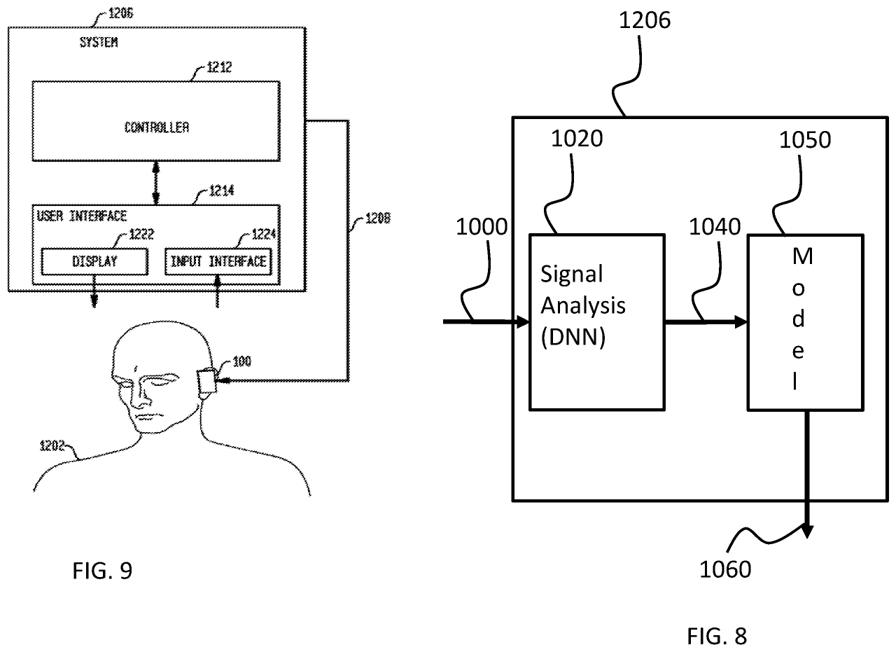

A system utilized to train a DNN or any other machine learning algorithm or system, along with acts associated therewith, is now described. The system will be described, at least in part, in terms of interaction with a recipient, although that term is used as a proxy for any pertinent subject to which the system is applicable (e.g., the test subjects used to train the DNN, the subject utilized to validate the trained DNN.). In an exemplary embodiment, system 1206, as seen in FIG. 8, is a recipient-controlled system while in other embodiments, it is a remote-controlled system. In an exemplary embodiment, system 1206 can correspond to a remote device and/or system, which, as detailed above, can be a portable handheld device (e.g., a smart device, such as a smart phone), and/or can be a personal computer, etc. In an exemplary embodiment, the system is under the control of an audiologist or the like, and subjects visit an audiologist center.

In an exemplary embodiment, the system can be a system having additional functionality according to the method actions detailed herein. In the embodiment illustrated in FIG. 9, any one or more of the devices disclosed herein can be connected to system 1206 to establish a data communication link 1208 between the device, such as the hearing prosthesis or such as the tinnitus mitigation device (where hereinafter, the phrase hearing prosthesis 100 is a proxy for any device that can enable the teachings detailed herein, such as a smartphone with a microphone, a dedicated microphone, a phone, etc., And thus the disclosure of a hearing prosthesis corresponds to a disclosure of another device as disclosed herein for linguistic economy) and system 1206. System 1206 is thereafter bi-directionally coupled by a data communication link 1208 with hearing prosthesis 100. Any communications link that will enable the teachings detailed herein that will communicably couple the implant and system can be utilized in at least some embodiments.

System 1206 can comprise a system controller 1212 as well as a user interface 1214. Controller 1212 can be any type of device capable of executing instructions such as, for example, a general or special purpose computer, a handheld computer (e.g., personal digital assistant (PDA)), digital electronic circuitry, integrated circuitry, specially designed ASICs (application specific integrated circuits), firmware, software, and/or combinations thereof. As will be detailed below, in an exemplary embodiment, controller 1212 is a processor. Controller 1212 can further comprise an interface for establishing the data communications link 1208 with the hearing prosthesis 100 (again, which is a proxy for any device that can enable the methods herein—any device with a microphone and/or with an input suite that permits the input data for the methods herein to be captured). In embodiments in which controller 1212 comprises a computer, this interface may be, for example, internal or external to the computer. For example, in an exemplary embodiment, controller 1206 and cochlear implant may each comprise a USB, FireWire, Bluetooth, Wi-Fi, or other communications interface through which data communications link 1208 may be established. Controller 1212 can further comprise a storage device for use in storing information. This storage device can be, for example, volatile or non-volatile storage, such as, for example, random access memory, solid state storage, magnetic storage, holographic storage, etc.

In an exemplary embodiment, input 1000 is provided into system 1206. The DNN signal analysis device 1020 analyzes the input 1000, and provides output 1040 to model section 1050, which establishes the model that will be utilized for the trained device. The output 1060 is thus the trained neural network, which is then uploaded onto the prosthesis or other component that is utilized to implement the trained neural network.

Here, the neural network can be “fed” statistically significant amounts of data corresponding to the input of a system and the output of the system (linked to the input), and trained, such that the system can be used with only input, to develop output (after the system is trained). This neural network used to accomplish this later task is a “trained neural network.” That said, in an alternate embodiment, the trained neural network can be utilized to provide (or extract therefrom) an algorithm or system that can be utilized separately from the trainable neural network. In one exemplary embodiment, a machine learning algorithm or a machine learning system starts off untrained, and then the machine learning algorithm or system is trained and “graduates” or matures into a usable product—the product of a trained machine learning system. With respect to another exemplary embodiment, the product from the trained machine learning—is the “offspring” of the trained machine learning (or some variant thereof, or predecessor thereof), which could be considered a mutant offspring or a clone thereof. That is, with respect to this second path, in at least some exemplary embodiments, the features of the machine learning system that enabled the machine learning system to learn may not be utilized in the practice of the first path, thus are not present in the first version. Instead, only the resulting product of the learning is used.

In an exemplary embodiment, the product from and/or of the machine learning utilizes non-heuristic processing to develop the data utilized in the trained system. In this regard, the system takes sound data or takes in general data relating to sound, and extracts fundamental signal(s) there from, and uses this to develop the model. By way of example only and not by way of limitation, the system utilizes algorithms beyond a first-order linear algorithm, and “looks” at more than a single extracted feature. Instead, the algorithm “looks” to a plurality of features. Moreover, the algorithm utilizes a higher order nonlinear statistical model, which self learns what feature(s) in the input is important to investigate. As noted above, in an exemplary embodiment, a DNN is utilized to achieve such. Indeed, in an exemplary embodiment, as a basis for implementing the teachings detailed herein, there is an underlying assumption that the features of the sound and other input into the system that enable the model to be generated may be too complex to be specified, and the DNN is utilized in a manner without knowledge as to what exactly on which the algorithm is basing its determinations/at which the algorithm is looking to develop the model.

In at least some exemplary embodiments, the DNN is the resulting product used to make the prediction. In the training phase there are many training operations algorithms which are used, which are removed once the DNN is trained.

To be clear, in at least some exemplary embodiments, the trained algorithm or system is such that one cannot analyze the trained algorithm or system with the resulting product therefrom to identify what signal features or otherwise what input features are utilized to produce the output of the trained neural network. In this regard, in the development of the system, the training of the algorithm or system, the system is allowed to find what is most important on its own based on statistically significant data provided thereto. In some embodiments, it is never known what the system has identified as important at the time that the system's training is complete. The system is permitted to work itself out to train itself and otherwise learn to control the prosthesis.

Briefly, it is noted that at least some of the neural networks or other machine learning systems utilized herein do not utilize correlation, or, in some embodiments, do not utilize simple correlation, but instead develop relationships. In this regard, the learning model is based on utilizing underlying relationships which may not be apparent or otherwise even identifiable in the greater scheme of things. In an exemplary embodiment, MatLAB, Buildo, etc., are utilized to develop the neural network. In at least some of the exemplary embodiments detailed herein, the resulting train system is one that is not focused on a specific speech feature, but instead is based on overall relationships present in the underlying statistically significant samples provided to the system during the learning process. The system itself works out the relationships, and there is no known correlation based on the features associated with the relationships worked out by the system.

The end result is a product which is agnostic to at least some ambient environment and/or physiological features. That is, the product of the trained neural network and/or the product from the trained neural network is such that one cannot identify what ambient environment and/or physiological features are utilized by the product to develop the production (the output of the system). The resulting arrangement is a complex arrangement of an unknown number of features of sound that are utilized. In embodiments utilizing code, the code is written in the language of a neural network, and would be understood by one of ordinary skill in the art to be such, as differentiated from a code that utilized specific and known features. That is, in an exemplary embodiment, the code looks like a neural network. This is also the case with the products detailed herein. The product looks like a neural network, and the person of skill would recognize such and be able to differentiate that from something that has other origins.

Consistent with common neural networks, there are hidden layers, and the features of the hidden layer are utilized in the process to predict the hearing impediments of the subject.

The various devices herein are subcomponents thereof, such as the processing units and/or the chips and/or the electronics packages/devices disclosed herein can utilize various commonly a barrel of all analysis techniques, or other techniques now known or later developed, to identify various markers in an input and may do so in real-time (e.g., continually or periodically as the hearing prosthesis receives the audio input). For example, the processing unit may apply various well known trainable classifier techniques, such as neural networks, Gaussian Mixture models, Hidden Markov models, and tree classifiers. These techniques can be trained to recognize particular characteristics. For instance, a tree classifier can be used to determine the presence of speech in audio input. Further, various ones of these techniques can be trained to recognize segments or quiet spaces between words, and to recognize the difference between male and female voices. Moreover, these techniques could be scaled in order of complexity based on the extent of available computation power.

Implementation of a classifier can be executed utilizing several stages of processing. In a two-stage classifier, for instance, the first stage is used to extract information from a raw signal representing the received input, which can be audio provided by the one or more microphones. This information can be anything from the raw audio signal itself, to specific features of the audio signal (“feature extraction”), such as pitch, modulation depth, etc. The second stage then uses this information to identify one or more probability estimates for a current class at issue.

In order for the second stage of this technique to work, there is utilitarian value in training the second stage. Training involves, by way of example, collecting a pre-recorded set of example outputs (“training data”) from the system to be classified, representing what engineers or others agree is a highest probability classification from a closed set of possible classes to be classified, such as audio of music or speech recorded through the prosthesis microphones. To train the second stage, this training data is then processed by the first stage feature extraction methods, and these first stage features are noted and matched to the agreed class. Through this design process, a pattern will ultimately be evident among all the feature values versus the agreed class collected. Well-known algorithms may then be applied to help sort this data and to decide how best to implement the second stage classifier using the feature extraction and training data available. For example, in a tree classifier, a decision tree may be used to implement an efficient method for the second stage.

As still another example, the processing unit may apply various well known speech recognition techniques to detect the extent of speech in the audio input. Those techniques may require significant computational power and may or may not be suitable for real-time analysis by prosthesis processing units without the assistance of an external processing unit for instance. However, continued developments in signaling processing technology and speech recognition algorithms may make actual speech recognition, including speaker recognition, more suitable for implementation by the processing unit of a hearing prosthesis.

Moreover, to facilitate carrying out this analysis in real-time, the processing unit may limit its analysis to identify key parameters as proxies for more complex characteristics or may generally estimate various ones of the characteristics rather than determining them exactly.

Data logging/data capture can be executed using any one or more of the teachings of PCT Application Publication No. WO 2020/021487, published on Jan. 30, 2020.

In general terms, the teachings of that application are frequently directed towards logging sound scenes and the auditory environment. Such can be utilized with the teachings herein vis-à-vis logging the ambient auditory environment. It is also noted that the teachings thereof can be modified to log and/or capture data indicative of the other types of features of the ambient environment, as well as logging/capturing data of physiological features. In this regard, the input systems would be modified to be input devices that can capture or otherwise obtain data associated with the other types of environments and physiological features (e.g., different sensors, such as those detailed herein and variations thereof), and then the data that is obtained via the input systems is recorded or otherwise transmitted in a manner consistent with the teachings of the 487 publication, albeit in a modified form as would be understood by the person of ordinary skill in the art to do so.

Now with reference to FIG. 10, teachings are provided that enable at least some of the methods and/or devices herein, in at least some embodiments, where there is a sound capture component and/or where captured sound is analyzed. In this regard, any one or more of the following teachings associated with FIG. 10 can be utilized with the captured sound, wherein the captured sound is ambient sound which can be the voice of the person of interest were a voice of people speaking to him or her or a voice that the person of interest wants to hear, etc.

It is explicitly noted that at least some exemplary embodiments include the teachings below when combined with the non-voice data logging detailed herein and/or the scene classification logging detailed herein. It is further explicitly noted that at least some exemplary embodiments include the teachings below without the aforementioned data logging.

FIG. 10 is a simplified block diagram of an exemplary prosthesis 12 or other device that can enable the teachings detailed herein (this can be a body carried device that is specially designed for the tinnitus mitigation strategies herein, and thus this is not necessarily a hearing prosthesis) operable in accordance with the present disclosure, which can correspond to any of the prostheses detailed herein and/or variations thereof, if only in a modified manner. As shown, the example hearing prosthesis 12 generally includes one or more microphones (microphone inputs) 14 for receiving audio input representing an audio environment of the prosthesis recipient (in an alternate embodiment, microphones 14 can instead be other types of sensors, such as body temperature sensors or pulse rate sensors or any the other sensors detailed herein or variations thereof or any other sensor that can enable monitoring/data capture of the various physiological and/or ambient conditions—element 14 can instead be a global positioning system receiver, or in addition to element 14 for that matter), optionally a processing unit 16 having a translation module 18 for translating a representation of the received audio input into stimulation signals, and stimulation means (one or more stimulation outputs) 20 for stimulating the physiological system of the recipient in accordance with the stimulation signals and thus in accordance with the received audio input.

It is noted that in an exemplary embodiment, the apparatus of FIG. 10 can be utilized to collect and/or capture any of the data that is disclosed herein as being collected and/or captured or otherwise logged, unless otherwise noted. That said, it is noted that any of the functionality associated with the device of FIG. 10 can be transferred to the device of 2140 detailed above, and/or a remote device, such as a remote device that is in signal communication with the prosthesis 100 and/or the device 2140 via element 259, etc., providing that the art enables such otherwise that such can be utilitarian. Accordingly, any disclosure herein of functionality of the device of FIG. 10 can correspond to a disclosure of a functionality of any other device disclosed herein or any other device that can implement the teachings detailed herein.

In this regard, in some embodiments, there is functional migration between the implant and the device 2140, and vice versa, and between either of these two and the remote device via element 259, which can be implemented according to any of the teachings of WO2016/207860, providing that such enables such.

This example hearing prosthesis may represent any of various types of hearing prostheses, including but not limited to those discussed above, and the components shown may accordingly take various forms. By way of example, if the hearing prosthesis is a hearing aid, the translation module 18 may include an amplifier that amplifies the received audio input, and the stimulation means 20 may include a speaker arranged to deliver the amplified audio into the recipient's ear. As another example, if the hearing prosthesis is a vibration-based hearing device, the translation module 18 may function to generate electrical stimulation signals corresponding with the received audio input, and the stimulation means 20 may include a transducer that delivers vibrations to the recipient in accordance with those electrical stimulation signals. And as yet another example, if the hearing prosthesis is a cochlear implant, the translation module 18 may similarly generate electrical signals corresponding with the received audio input, and the stimulation means 20 may include an array of electrodes that deliver the stimulation signals to the recipient's cochlea. Other examples are possible as well.

In practice, the processing unit 16 may be arranged to operate on a digitized representation of the received audio input as established by analog-to-digital conversion circuitry in the processing unit, microphone(s) or one or more other components of the prosthesis. As such, the processing unit 16 may include data storage (e.g., magnetic, optical or flash storage) 22 for holding a digital bit stream representing the received audio and for holding associated data. Further, the processing unit 16 may include a digital signal processor, and the translation module 18 may be a function of the digital signal processor, arranged to analyze the digitized audio and to produce corresponding stimulation signals or associated output. Alternatively or additionally, the processing unit may include one or more general purpose processors (e.g., microprocessors), and the translation module 18 may include a set of program instructions stored in the data storage 22 and executable by the processor(s) to analyze the digitized audio and to produce the corresponding stimulation signals or associated output.

As further shown, the example hearing prosthesis 12 includes or is coupled with a user interface system 24 through which the recipient or others (e.g., a clinician) may control operation of the prosthesis and view various settings and other output of the prosthesis. In practice, for instance, the user interface system 24 may include one or more components internal to or otherwise integrated with the prosthesis. Further, the user interface system 24 may include one or more components external to the prosthesis, and the prosthesis may include a communication interface arranged to communicate with those components through a wireless and/or wired link of any type now known or later developed.

In a representative arrangement, the user interface system 22 may include one or more user interface components that enable a user to interact with the hearing prosthesis. As shown by way of example, the user interface components may include a display screen 26 and/or one or more input mechanisms 28 such as a touch-sensitive display surface, a keypad, individual buttons, or the like. These user interface components may communicate with the processing unit 16 of the prosthesis in much the same way that conventional user interface components interact with the host processor of a personal computer. Alternatively, the user interface system 24 may include one or more standalone computing devices such as a personal computer, mobile phone, tablet, handheld remote control, or the like, and may further include its own processing unit 30 that interacts with the prosthesis and may be arranged to carry out various other functions.

In practice, user interface system 24 may enable the recipient to control the stimulation mode of the hearing prosthesis, such as to turn stimulation functionality on and off. For instance, at times when the recipient does not wish to have the prosthesis stimulate the recipient's physiological system in accordance with received audio input, the recipient may engage a button or other input mechanism of the user interface system 24 to cause processing unit 16 to set the prosthesis in the stimulation-off mode. And at times when the recipient wishes to have the prosthesis stimulate the recipient's physiological system in accordance with the received audio input, the recipient may engage a similar mechanism to cause the processing unit 16 to set the prosthesis in the stimulation-on mode. Further, the user interface system 24 may enable the recipient or others to program the processing unit 16 of the prosthesis so as to schedule automatic switching of the prosthesis between the stimulation-on mode and the stimulation-off mode.

In accordance with the present disclosure, as noted above, the example hearing prosthesis 12 will additionally function to log and output data regarding the received audio input. The hearing prosthesis may then output logged data from time to time for external analysis, and/or can be analyzed with a device that is part of the prostheses in at least some embodiments.

The audio input that forms the basis for this analysis is the same audio input that the hearing prosthesis is arranged to receive and use as a basis to stimulate the physiological system of the recipient when the prosthesis is in the stimulation-on mode. Thus, as the prosthesis receives audio input, the prosthesis may not only translate that audio input into stimulation signals to stimulate the recipient's physiological system if the hearing prosthesis is in the stimulation-on mode but may also log data regarding the same received audio output, such as data regarding linguistic characteristics in the audio input in correlation with the stimulation mode. Further, even at times when the hearing prosthesis is receiving audio input but is not stimulating the recipient's physiological system (e.g., because stimulation is turned off or because the audio input amplitude or frequency is such that the prosthesis is set to not provide stimulation), the prosthesis may still log data regarding that received audio input, such as linguistic characteristics in correlation with the stimulation mode. Any or all of this data may then be clinically relevant and useful in developing mediation for the recipient.

It is also noted that the machine learning and/or data collection and/or data capture features and/or data analysis features detailed herein can be executed via any one or more of the teachings of PCT patent application publication no. 2018/087674, published on May 17, 2020, providing that the art enables such.

It is noted that any method detailed herein also corresponds to a disclosure of a device and/or system configured to execute one or more or all of the method actions associated therewith detailed herein. In an exemplary embodiment, this device and/or system is configured to execute one or more or all of the method actions in an automated fashion. That said, in an alternate embodiment, the device and/or system is configured to execute one or more or all of the method actions after being prompted by a human being. It is further noted that any disclosure of a device and/or system detailed herein corresponds to a method of making and/or using that the device and/or system, including a method of using that device according to the functionality.

Any action disclosed herein that is executed by the prosthesis 100 or the prosthesis of FIG. 2 or the device of FIG. 2C or any other device disclosed herein can be executed by the device 2140 and/or another component of any system detailed herein in an alternative embodiment, unless otherwise noted or unless the art does not enables such. Thus, any functionality of the prosthesis 100 with the prosthesis of FIG. 2 or the device of FIG. 2C, etc. can be present in the device 2140 and/or another component of any system in an alternative embodiment. Thus, any disclosure of a functionality of the prosthesis 100 or the other prostheses detailed herein and/or the other devices disclosed herein corresponds to structure of the device 2140 and/or the another component of any system detailed herein that is configured to execute that functionality or otherwise have a functionality or otherwise to execute that method action.

Any action disclosed herein that is executed by the device 2140 can be executed by the prosthesis 100 or any of the other devices such as the prostheses of FIG. 2 and/or the prosthesis of figure to say and/or another component of any system disclosed herein in an alternative embodiment, unless otherwise noted or unless the art does not enables such. Thus, any functionality of the device 2140 can be present in the prosthesis 100 or any the other devices disclosed herein, such as the devices of FIG. 2 and/or FIG. 2C and/or another component of any system disclosed herein in an alternative embodiment. Thus, any disclosure of a functionality of the device 2140 corresponds to structure of the prosthesis 100 or any other device disclosed herein and/or another component of any system disclosed herein that is configured to execute that functionality or otherwise have a functionality or otherwise to execute that method action.

Any action disclosed herein that is executed by a component of any system disclosed herein can be executed by the device 2140 and/or the prosthesis 100 or the prosthesis of FIG. 2 or the device of figure to say in an alternative embodiment, unless otherwise noted or unless the art does not enables such. Thus, any functionality of a component of the systems detailed herein can be present in the device 2140 and/or the prosthesis 100 and/or the other devices disclosed herein, such as the device of FIG. 2 and/or the device of FIG. 2C as alternative embodiment. Thus, any disclosure of a functionality of a component herein corresponds to structure of the device 2140 and/or the prosthesis 100 and/or the device of FIG. 2 and/or the device of FIG. 2C that is configured to execute that functionality or otherwise have a functionality or otherwise to execute that method action. It is further noted that any disclosure of a device and/or system detailed herein also corresponds to a disclosure of otherwise providing that device and/or system.

It is also noted that any disclosure herein of any process of manufacturing other providing a device corresponds to a device and/or system that results there from. It is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any disclosure herein of a method action corresponds to a disclosure of a computer readable medium having program there on code to execute one or more of those actions and also a product to execute one or more of those actions.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1-10. (canceled)

11. An apparatus, comprising:
a body carried portable device including an input subsystem and an output subsystem, wherein
the device includes a product of and/or resulting from machine learning that is used by the device to determine when and/or if to initiate a tinnitus management action.

12. The apparatus of claim 11, wherein:
the product of an/or resulting from machine learning is also used by the device to determine what type of tinnitus management action should be executed based on input into the input subsystem, wherein the management action at least one of remediates the effects of tinnitus or prevents a noticeable tinnitus scenario from occurring.

13. The apparatus of claim 11, wherein:
the input subsystem is configured to automatically obtain data indicative of at least physiological features past and/or present of a person who is using the device for tinnitus management purposes; and
the input into the subsystem is the obtained data.

14. The apparatus of claim 11, wherein:
the input subsystem is configured to automatically obtain data indicative of at least ambient environmental conditions past and/or present of a person who is using the device for tinnitus management purposes; and
the input into the subsystem is the obtained data.

15. The apparatus of claim 11, wherein:
the input subsystem is configured to automatically obtain data indicative of speech in an ambient environment;
the device is configured to analyze the input and determined that the speech is likely speech that a user of the device seeks to understand; and
the device automatically adjusts a tinnitus therapy based on the analysis.

16. The apparatus of claim 11, wherein:
the device is configured to automatically initiate tinnitus masking using the product based on the input into the input subsystem.

17. The apparatus of claim 11, wherein:
the device is configured to log data indicative of at least one of ambient environmental conditions past and/or present of a person who is using the device for tinnitus management purposes or ambient environmental conditions past and/or present of a person who is using the device for tinnitus management purposes; and
the device is configured to correlate the logged data to tinnitus related events.

18-26. (canceled)

27. A system, comprising:
a sound capture apparatus configured to capture ambient sound; and
an electronics package configured to receive data based on at least an outputted signal from the sound capture apparatus and analyze the data to determine based thereon that there exists a statistical likelihood of a future tinnitus event in the near term of a person using the system, wherein
the system is configured to automatically initiate an output that preemptively reduces the likelihood of the future tinnitus event upon the determination.

28. The system of claim 27, wherein:
the system is configured to automatically initiate the output without affirmative input from the person.

29. The system of claim 27, wherein:
the data received by the electronics package further includes data based on physiological data relating to the person; and
the electronics package is configured to evaluate the data based on physiological data in combination with the data based on the outputted signal an determine based thereon that there exists a statistical likelihood of a future tinnitus event in the near term of a person using the system.

30. The system of claim 27, wherein:
the system includes a hearing prosthesis, the hearing prosthesis including the sound capture device.

31. The system of claim 27, wherein:
the electronics package includes logic that applies a dynamic and individualized probability metric to determine that there exists the statistical likelihood of a future tinnitus event in the near term of a person using the system.

32. The system of claim 27, wherein:
the system is configured to automatically log data indicative of at least one of ambient environmental conditions past and/or present of the person or physiological conditions past and/or present of the person;
the system is configured to automatically correlate the logged data to tinnitus related events of the person and automatically develop a tinnitus management regime; and
the electronics package is configured to execute the tinnitus management regime to analyze the data to determined based on the data that there exists the statistical likelihood of the future tinnitus event in the near term of the person using the system.

33. The system of claim 27, wherein:
the ambient environmental conditions include the presence of speech.

34. A system, comprising:
a tinnitus onset predictive subsystem; and
a tinnitus management output subsystem.

35. The system of claim 34, wherein:
the system further comprises a tinnitus onset predictive metric development subsystem.

36. The system of claim 35, wherein:
the system includes a trained neural network, wherein the trained neural network is part of the tinnitus onset predictive subsystem; and
the tinnitus onset predictive metric development subsystem contributes to the training of the trained neural network.

37. The system of claim 34, wherein:
the tinnitus onset predictive subsystem is an expert sub-system of the system that includes a code of and/or from a machine learning algorithm to analyze data relating to a user of the system in real time and wherein the machine learning algorithm is a trained system trained based on a statistically significant population of tinnitus afflicted persons.

38. The system of claim 34, wherein:
the tinnitus onset predictive subsystem is configured to automatically analyze a linguistic environment metric in combination with a non-linguistic environment metric correlated to the linguistic environment metric, all inputted into the system, and based on the analysis, automatically determine whether or not a tinnitus event is imminent.

39. The system of claim 38, wherein:
the system is configured to identify speech of a user of the system; and
the linguistic environment metric is the speech of the user.

40. The system of claim 34, wherein:
the tinnitus management output subsystem diverts a user of the system's attention, thus mitigating the effects of tinnitus.

41-43. (canceled)
